(19) United States
(12) Patent Application Publication
(10) Pub. No.: US 2023/0019900 A1
(43) Pub. Date: Jan. 19, 2023

(54) PREDICTION OF VENOUS THROMBOEMBOLISM UTILIZING MACHINE LEARNING MODELS

(71) Applicants: Henry M. Jackson Foundation for the Advancement of Military Medicine, Bethesda, MD (US); The Government of the United States, as represented by the Secretary of the Army, Fort Detrick, MD (US); The United States of America, as Represented by the Secretary of the Navy, Silver Spring, MD (US)

(72) Inventors: Matthew J. Bradley, Silver Spring, MD (US); Eric A Elster, Kensington, MD (US); Vivek Khatri, Bethesda, MD (US); John S Oh, Fort Detrick, MD (US); Seth A Schobel, Clarksburg, MD (US)

(73) Assignees: Henry M. Jackson Foundation for the Advancement of Military Medicine, Bethesda, MD (US); The Government of the United States, as represented by the Secretary of the Army, Fort Detrick,, MD (US); The United States of America, as Represented by the Secretary of the Navy, Silver Spring, MD (US)

(21) Appl. No.: 17/756,805

(22) PCT Filed: Dec. 3, 2020
(86) PCT No.: PCT/US2020/063107

§ 371 Date: Jun. 2, 2022

Related U.S. Application Data
(60) Provisional application No. 62/944,836, filed on Dec. 6, 2019.

(51) Int. Cl. G16H 50/20; G16B 40/00

(52) U.S. Cl. CPC G16H 50/20; G16B 40/00

(57) ABSTRACT

The present disclosure describes methods and systems for predicting if a subject has an increased risk of having or developing venous thromboembolism, including prior to the detection of symptoms thereof and/or prior to onset of any detectable symptoms thereof. The present disclosure also describes a method of generating a model for predicting venous thromboembolism.

### CROSS REFERENCE TO RELATED APPLICATIONS

This application claims the benefit of U.S. Provisional Application No. 62/944,836 filed Dec. 6, 2019, entitled Prediction of Venous Thromboembolism Utilizing Machine Learning Models”, which is incorporated herein by reference in its entirety.

### STATEMENT REGARDING FEDERALLY SPONSORED RESEARCH OR DEVELOPMENT

This invention was made with government support under grant USUHS HT9404-13-1-0032 and HU0001-15-2-0001 awarded by the Department of Defense and Defense Health Program. The government has certain rights in the invention.

### FIELD OF THE DISCLOSURE

Described herein are systems, methods, and computational environments for predicting venous thromboembolism in a subject having an injury based on clinical parameters and biological data. Also described are systems and methods for predicting venous thromboembolism for a subject, systems and methods for generating multiple models for predicting venous thromboembolism and determining the most accurate model, and methods of treating a subject determined to have an elevated risk of a specific outcome, methods of detecting panels of biomarkers in a subject, and methods of assessing risk factors in a subject having an injury.

### BACKGROUND OF THE DISCLOSURE

Individuals who are exposed to physical trauma (e.g. on the battlefield or major accidents) have elevated risks of injuries, including venous thromboembolism. The myriad potential injuries and the lack of ability to perform robust, invasive diagnostics (e.g. MRI, CT-scans, angiography) make it prohibitively difficult to accurately quantify risk of venous thromboembolism and make high-fidelity decisions on treatment and intervention strategies. Having tools that would allow a clinician, either in the field or at the bedside, to predict or identify the patients at highest risk for a variety of complications could allow for more proactive and directed preventative strategies.

### SUMMARY OF THE DISCLOSURE

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

Described herein are methods of determining if a subject has an increased risk of having or developing venous thromboembolism, including prior to the detection of symptoms thereof and/or prior to onset of any detectable symptoms thereof, methods for predicting venous thromboembolism, and related methods of treatment. In embodiments, there are provided methods for predicting venous thromboembolism for a subject comprising, generating a training database storing first values of a plurality of clinical parameters and venous thromboembolism associated with a plurality of first subjects; formatting the database into model features configured to be input into the machine learning model; executing a feature selection algorithm to select a subset of model parameters from the plurality of clinical parameters for the machine learning model; inputting the selected subset of features into the machine learning models for predicting venous thromboembolism; generating, utilizing at least the machine learning models for predicting venous thromboembolism, output data indicating a prediction for venous thromboembolism; and calculating a performance metric associated with the machine learning model in accordance with the prediction of venous thromboembolism.

In embodiments, there are provided methods for predicting a clinical outcome for a subject comprising, receiving, from a second subject, a second value of at least one clinical parameter of a plurality of clinical parameters; executing a pre-trained model for predicting venous thromboembolism, wherein the model is pre-trained by performing operations comprising: generating a training database storing first values of a plurality of clinical parameters and venous thromboembolism associated with a plurality of first subjects; formatting the database into model features configured to be input into the machine learning model; executing a feature selection algorithm to select a subset of model parameters from the plurality of clinical parameters for the machine learning model; inputting the selected subset of features into the machine learning models for predicting venous thromboembolism; generating, utilizing at least the machine learning models for predicting venous thromboembolism, output data indicating a prediction for venous thromboembolism; and calculating a performance metric associated with the machine learning model in accordance with the prediction of venous thromboembolism; and outputting the predicted venous thromboembolism of the second subject.

In embodiments, there are provided methods comprising pre-processing data that is stored in the training database including: determining that a first value of at least one of the plurality of clinical parameters is missing; estimating a reference value for the at least one of the plurality of clinical parameters that is missing; and storing the reference value as the first value of the at least one of the plurality of clinical parameters in the training database.

In embodiments, the plurality of feature selection machine learning models comprise at least one of unsupervised machine learning algorithm, supervised machine learning algorithm, univariate t-tests, backwards elimination, and recursive feature elimination. While these algorithms are enumerated for feature selection machine learning, many others are contemplated.

In embodiments, the algorithm is a supervised machine learning algorithm and the machine learning model for predicting venous thromboembolism comprises a random forest model.

In embodiments, there are provided methods for cross-validating performances of the machine learning model, wherein cross-validating comprises iterations of leave-one-pair-out cross validation.

Cross-validation is an approach to test a model's ability to predict new data that was not used in the estimation (training) of the model. Cross-validation is often used by comparing the results from one subset of a data set to another subset of the data set. For example, leave on out cross-validation is a method of cross-validation wherein one data point is removed to test the model. As another example, K-fold validation is similar to leave one out cross-validation, but the dataset is divided into k subsets, where k represents an integer; the cross-validation is repeated k times and each time one of the k subsets is used as the test set and the other k−1 subsets are put together to form a training set, then the average error across all k trials is computed. While these methods are enumerated, this list is not exhaustive and many other forms of cross-validation are contemplated

In embodiments, the performance metric associated with each of the plurality of prediction algorithms includes at least one of a total out-of-bag (OOB) error estimate, a positive class OOB error estimate, a negative OOB error estimate, an accuracy score, area under the curve (AUC) measure from a receiver operating characteristic (ROC) curve, sensitivity, specificity, or a Kappa score.

In embodiments, the plurality of clinical parameters comprise one or more of subject data, administration of blood products data, and injury severity data, or a combination thereof.

In embodiments, there are provided systems for generating a machine learning engine for predicting venous thromboembolism in a subject comprising: one or more processors; a memory; a communication platform; a training database configured to store first values of a plurality of clinical parameters and venous thromboembolism associated with a plurality of first subjects; a machine learning engine configured to execute a plurality of feature selection machine learning models to select a subset of model parameters from the plurality of clinical parameters for each feature selection machine learning models; execute each one of a plurality of prediction machine learning models for one of the plurality of subsets of model parameters to generate predictions of the venous thromboembolism; calculate a performance metric associated with each of the plurality of prediction machine learning models in accordance with the predictions of the venous thromboembolism; select a candidate prediction machine learning model in accordance with the performance metric; and output a machine learning model for predicting venous thromboembolism, the machine learning model comprising the candidate classification machine learning model with associated subset of model parameters.

In embodiments, there are provided systems for predicting venous thromboembolism in a subject comprising: one or more processors; a memory; a communication platform; a training database configured to store first values of a plurality of clinical parameters and venous thromboembolism outcomes associated with a plurality of first subjects; and a machine learning engine configured to: format the database into model features configured to be input into a machine learning model; execute a feature selection algorithm to select a subset of model parameters from the plurality of clinical parameters for the machine learning model; input the selected subset of features into the machine learning models for predicting venous thromboembolism; generate, utilizing at least the machine learning models for predicting venous thromboembolism, output data indicating a prediction for venous thromboembolism; calculate a performance metric associated with the machine learning model in accordance with the prediction of venous thromboembolism; output a trained machine learning model for predicting venous thromboembolism; receive, from a second subject, a second value of at least one clinical parameter of a plurality of clinical parameters; execute the trained model for predicting venous thromboembolism; and output data indicating a prediction for venous thromboembolism on a display device.

There are provided methods for predicting venous thromboembolism in a subject comprising: obtaining a biological sample from the subject; measuring clinical parameters; and predicting venous thromboembolism in the subject, based at least in part on the measured levels of clinical parameters. In embodiments, the clinical parameters include one or more of IL-15, MIG, VEGF, total number of blood products transfused in first 24 hours, or assessing soft tissue injury. In embodiments, predicting venous thromboembolism is based in part on measuring clinical parameters comprising one or more of IL-15, MIG, or VEGF.

### BRIEF DESCRIPTION OF THE SEVERAL VIEWS OF THE DRAWINGS

The present disclosure can be better understood by reference to the following drawings. The drawings are merely exemplary to illustrate certain features that may be used singularly or in combination with other features and the present disclosure should not be limited to the embodiments shown.

FIG. 1 depicts a method of predicting venous thromboembolism through a process of generating of training data, formatting data, executing feature selection algorithms, inputting features into machine learning models, generating output data indicating predictions for venous thromboembolism, and calculating performance metrics.

FIG. 2 illustrates a block diagram for a venous thromboembolism prediction system for an individual as described herein.

FIG. 3 illustrates a flow-chart for an embodiment of the invention.

FIG. 4 illustrates an embodiment of a computational environment that involves a computing device, a network, and a remote device.

FIG. 5 show the results of the performance of exemplary models (Random Forest models) with various features and their respective performance metrics.

### DETAILED DESCRIPTION

The following detailed description is presented to enable any person skilled in the art to make and use the subject of the application. For purposes of explanation, specific nomenclature is set forth to provide a thorough understanding of the present disclosure. However, it will be apparent to one skilled in the art that these specific details are not required to practice the subject of the application. Descriptions of specific applications are provided only as representative examples. The present application is not intended to be limited to the embodiments shown but is to be accorded the widest possible scope consistent with the principles and features disclosed herein.

The present disclosure provides methods of predicting venous thromboembolism for individuals determined to have an increased risk of developing one or more of the venous thromboembolism, optionally before the onset of detectable symptoms thereof, such as before there are perceivable, noticeable, or measurable signs of venous thromboembolism in the individual. The individual may be undergoing established treatment and based on the clinical outcome predicted by the methods described herein, adjustment can be made for more appropriate treatment.

Established venous thromboembolism treatments include anticoagulants, including injectables such as heparin or low molecular weight heparin, or tablets such as apixaban, dabigatran, rivaroxaban, edoxaban and warfarin. Other treatments can be included given the comorbidities that occur with venous thromboembolism, such as: acute kidney injury, acute respiratory distress, bacteremia, heterotopic ossification, pneumonia, post-traumatic sterile inflammation, sepsis, wound closure, or vasospasm and/or mortality following traumatic brain injury, or a combination thereof. The traumatic brain injury can be a severe TBI (sTBI) or a mild TBI. Established acute kidney injury treatments include balancing fluids and electrolytes in addition to renal replacement therapy. Established acute respiratory distress treatments include fluid management and adjustment of mechanical ventilation to optimize tissue oxygenation and minimize hypoxia. Established bacteremia treatments include the adjustment of antibiotic treatment. Established heterotopic ossification treatments include its prevention in part by early use of nonsteroidal anti-inflammatory drugs. Established pneumonia treatments include the adjustment of antibiotic treatment. Established post-traumatic sterile inflammation with systemic inflammation treatments include a multiple assessment of biochemical, metabolic and hemodynamic profiles for individual adjustments in medication, fluid and electrolytes. Established sepsis treatment includes an immediate antibiotic treatment combined with a hemodynamic and electrolyte balance adjustments. Established TBI-induced vasospasm treatments include calcium channel blocker medications to prevent further associated complications. Established wound closure treatments include an adequate adjustment for the number of surgical debridements to allow successful healing by delayed primary closure. While these treatments for venous thromboembolism are discussed, many more are contemplated. Benefits of such early treatment may include reduced risk of complications of venous thromboembolism, avoidance of sepsis, empyema, need for ventilation support, reduced length of stay in hospital or intensive care unit, reduced mortality, lower risk of limb loss, and/or reduced medical costs.

The present disclosure also provides using the methods described herein to monitor subjects to help clinicians make decisions on adjusting treatments, when necessary.

Technical and scientific terms used herein have the meanings commonly understood by one of ordinary skill in the art to which the present disclosure pertains, unless otherwise defined.

As used herein, the singular forms “a,” “an,” and “the” designate both the singular and the plural, unless expressly stated to designate the singular only.

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject, and is not limited to any specific dosage forms or routes of administration unless otherwise stated.

The terms “treat”, “treating” or “treatment”, as used herein, include alleviating, abating or ameliorating venous thromboembolism or one or more symptoms thereof, whether or not venous thromboembolism is considered to be “cured” or “healed” and whether or not all symptoms are resolved. The terms also include reducing or preventing progression of venous thromboembolism, including venous thromboembolism, or one or more symptoms thereof, impeding or preventing an underlying mechanism of venous thromboembolism or one or more symptoms thereof, and achieving any therapeutic and/or prophylactic benefit.

As used herein, the term “subject,” “subject in need thereof,” “patient,” “individual,” or “test subject” indicates a mammal, in particular a human or non-human primate. The test subject may or may not be in need of an assessment of a predisposition to venous thromboembolism. In embodiments, the test subject is assessed prior to the detection of symptoms of venous thromboembolism. In embodiments, the test subject is assessed prior to the onset of any detectable symptoms of venous thromboembolism. In embodiments, the test subject does not have detectable symptoms of any type of sickness or condition. In embodiments, the test subject has an injury, condition, or wound that puts the subject at risk of developing venous thromboembolism, such as having a viral or bacterial infection, such as but not limited to urinary tract infection, meningitis, pericarditis, endocarditis, osteomyelitis, and infectious arthritis, having or developing bronchitis, undergoing a medical surgical or dental procedure, having an open wound or trauma, such as but not limited to a wound received in combat, a blast injury, a crush injury, a gunshot wound, an extremity wound, suffering a nosocomial infection, having undergone medical interventions such as central line placement or intubation, having diabetes, having HIV, undergoing hemodialysis, undergoing organ transplant procedure (donor or receiver), receiving a glucocorticoid or any other immunosuppressive treatments, such as but not limited to calcineurin inhibitors, mTOR inhibitors, IMDH inhibitors and biologics or monoclonal antibodies. In embodiments, the subject does not have a condition that puts the subject at risk of developing venous thromboembolism, prior to application of the methods described herein. In embodiments, the subject has a condition that puts the subject at risk of developing venous thromboembolism.

As used herein, the term “increased risk” or “elevated risk” is used to mean that the test subject has an increased chance of developing or acquiring venous thromboembolism compared to a normal or reference individual or population of individuals. In embodiments, the reference individual is the test subject at an earlier time point, including prior to having an injury, condition, or wound that puts the subject at risk of developing venous thromboembolism, or at an earlier point in time after having such an injury, condition, or wound. The increased risk may be relative or absolute and may be expressed qualitatively or quantitatively. For example, an increased risk may be expressed as simply determining the subject's risk profile and placing the subject in an “increased risk” category, based upon previous studies. Alternatively, a numerical expression of the subject's increased risk may be determined based upon the risk profile. As used herein, examples of expressions of an increased risk include but are not limited to, odds, probability, odds ratio, p-values, attributable risk, biomarker index score, relative frequency, positive predictive value, negative predictive value, and relative risk. Risk may be determined based on predicting venous thromboembolism for the subject; for example, a predicted venous thromboembolism outcome may include an indication of whether the subject has venous thromboembolism or does not have venous thromboembolism, an indication of a likelihood that the subject has venous thromboembolism or does not have venous thromboembolism, or an indication of a likelihood that the subject will develop venous thromboembolism.

For example, the correlation between a subject's risk profile and the likelihood of suffering from venous thromboembolism may be measured by an odds ratio (OR) and by the relative risk (RR). If P(R⁺) is the probability of developing venous thromboembolism for individuals with the risk profile (R) and P(R⁻) is the probability of developing venous thromboembolism for individuals without the risk profile, then the relative risk is the ratio of the two probabilities: RR=P(R⁺)/P(R⁻).

In case-control studies, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of a risk profile in cases studies and F⁻ is the frequency of risk profile in controls. F⁺ and F⁻ can be calculated using the risk profile frequencies of the study.

The attributable risk (AR) can also be used to express an increased risk. The AR describes the proportion of individuals in a population exhibiting a specific clinical outcome, such as venous thromboembolism, to a specific member of the risk profile. AR may also be important in quantifying the role of individual components (specific member) in condition etiology and in terms of the public health impact of the individual risk factor. The public health relevance of the AR measurement lies in estimating the proportion of cases of the specific clinical outcome in the population that could be prevented if the profile or individual factor were absent. AR may be determined as follows: AR=P_{E}(RR−1)/(P_{E}(RR−1)+1), where AR is the risk attributable to a profile or individual factor of the profile, and P_{E} is the frequency of exposure to a profile or individual component of the profile within the population at large. RR is the relative risk, which can be approximated with the odds ratio when the profile or individual factor of the profile under study has a relatively low incidence in the general population.

The terms “factor,” “risk factor,” and/or “component” are used herein to refer to individual constituents that are assessed, detected, measured, received, and/or determined prior to or during the performance of any of the methods described herein. For convenience, they are referred to herein as clinical parameters.

Clinical parameters include various factors associated with a subject experiencing symptoms of a disease or condition or in measurable changes in health, function, or quality of life. Examples of clinical parameters of a subject include, but are not limited to any one or more of level of interleukin-1α (IL-1α) in a sample from the subject, level of interleukin-Iβ (IL-Iβ) in a sample from the subject, level of interleukin-1 receptor agonist (IL-1RA) in a sample from the subject, level of interleukin-2 (IL-2) in a sample from the subject, level of interleukin-2 receptor (IL-2R) in a sample from the subject, level of interleukin-3 (IL-3) in a sample from the subject, level of interleukin-4 (IL-4) in a sample from the subject, level of interleukin-5 (IL-5) in a sample from the subject, level of interleukin-6 (IL-6) in a sample from the subject, level of interleukin-7 (IL-7) in a sample from the subject, level of interleukin-8 (IL-8) in a sample from the subject, level of interleukin-10 (IL-10) in a sample from the subject, level of interleukin-12 (IL-12) in a sample from the subject, level of interleukin-13 (IL-13) in a sample from the subject, level of interleukin-15 (IL-15) in a sample from the subject, level of interleukin-17 (IL-17) in a sample from the subject, level of tumor necrosis factor alpha (TNF-α) in a sample from the subject, level of granulocyte colony stimulating factor (G-CSF) in a sample from the subject, level of granulocyte macrophage colony stimulating factor (GM-CSF) in a sample from the subject, level of interferon alpha (IFN-α) in a sample from the subject, level of interferon gamma (IFN-γ) in a sample from the subject, level of epithelial growth factor (EGF) in a sample from the subject, level of basic fibroblast growth factor (bFGF) in a sample from the subject, level of hepatocyte growth factor (HGF) in a sample from the subject, level of vascular endothelial growth factor (VEGF) in a sample from a subject, the level of monocyte chemoattractant protein-1 (CCL2/MCP-1) in a sample from a subject, the level of macrophage inflammatory protein-1 alpha (CCL3/MIP-Iα) in a sample from a subject, the level of macrophage inflammatory protein-1 beta (CCl-4/MIP-Iβ) in a sample from a subject, the level of CCL5/RANTES in a sample from a subject, the level of CCLII/eotaxin in a sample from a subject, the level of monokine induced by gamma interferon (CXCL9/MIG) in a sample from a subject, the level of interferon gamma-induced protein-10 (CXCL10/IP10) in a sample from a subject, the level of mitochondrial DNA (mtDNA) in a sample from a subject, the level of soluble CD40 ligand (sCD40L) in a subject, the level of transglutaminase 2 in a sample from a subject, gender, age, date of injury, location of injury, presence of abdominal injury, mechanism of injury, wound depth, wound surface area, number of wound debridements, associated injuries, type of wound closure, success of wound closure, requirement for transfusion, total number of blood products transfused, amount of whole blood cells administered to the subject, amount of red blood cells (RBCs) administered to the subject, units of blood products transfused in the first 24 hours, amount of packed red blood cells (pRBCs) administered to the subject, amount of platelets administered to the subject, level of total packed RBCs, Injury Severity Score (ISS), Abbreviated Injury Scale (AIS) of head, AIS of abdomen, AIS of chest (thorax), Acute Physiology and Chronic Health Evaluation II (APACHE II) score, location of injury, presence of abdominal injury, mechanism of injury, wound depth, wound surface area, number of wound debridements, associated injuries, type of wound closure, success of wound closure, wound presence and location, compound fracture, soft tissue injury, limb amputation, presence of critical colonization (CC) in a sample from the subject, presence of traumatic brain injury, severity of traumatic brain injury, length of hospital stay, length of intensive care unit (ICU) stay, number of days on a ventilator, disposition from hospital, development of nosocomial infections, sequential organ failure assessment, injury GCS score, Marshall Classification 2 (mild diffuse injury), midline shift based on Rotterdam Computed Tomography (Rott CT)s, temperature, arterial pH, pulse rate, and FiO₂.

While one or more of these markers may be applied to venous thromboembolism, subsets of these markers may provide more predictive power toward venous thromboembolism. Markers and the associated models and their respective prognostic capacity as measured by performance metrics are noted in table 1, wherein venous thromboembolism is included. The markers in table 1 provide exemplary embodiments of the application of machine learning models using different machine learning models for predicting diseases or conditions. One or more of the markers or a combination of all the markers listed in Table 1 for each respective clinical outcome can be used to predict each of the clinical outcome. One or more includes two or more, three or more, four or more, etc. of the markers. The markers could be obtained from various biological samples and could be proteins or nucleic acids.

TABLE 1
List of markers and machine learning models tested by clinical outcome

| Clinical Outcome | Markers | Machine Learning Models | Performance Metrics |
|---|---|---|---|
| Venous Thromboembolism | IL-15, MIG, VEGF, Units of Total Blood | Random Forest, Leave One | AUC—0.946, Sensitivity— |
| | Products Transfused-first 24 hr, Soft | Out Cross-Validation, | 0.992, Specificity—0.838, |
| | Tissue Injury | Backwards Elimination | Threshold—0.22 |
| Acute Kidney Injury after Trauma | Sequential Organ Failure Assessment | Logistic Regression, Random | Random Forest: AUC— |
| Laparotomy | (SOFA) score, serum MCP-1, serum | Forest | 0.74, Sensitivity—0.82, |
| | VEGF | | Specificity—0.61 |
| | | | Logistic Regression: AUC— |
| | | | 0.72, Sensitivity—0.77, |
| | | | Specificity—0.64 |
| Acute Kidney Injury in Post- | ISS, APACHE II, blood transfusion | Leave One Out Cross | AUC—0.93, Sensitivity— |
| Trauma Combat Patients | product, nosocomial infection, wound | Validation, Backwards | 0.91, Specificity—0.91 |
| | size, and serum levels of IL-2R, IL-6, | Elimination | |
| | and MCP-1 | | |
| Acute Respiratory Distress | APACHE potassium score, injury GCS | Bayesian Belief Networks, 5- | AUC—0.88, Sensitivity— |
| | score verbal, IS head, total SOFA and | Fold Cross Validation, | 0.92, Specificity—0.88 |
| | serum procalcitonin (ProCT) | Receiver Operating Curve | |
| Bacteremia | 1. ISS, MIG, IL-6, IL-7, IL-8 | Backwards Elimination, | Model 1: AUC—0.82, |
| | 2. Serum biomarkers: GCSF, GMCSF, | Leave One Out Cross | Sensitivity—0.79, Specificity— |
| | IL-1 a, IL-1B, IL-2, IL-4, IL-6, IL-8 VEGF | Validation, Random Forest, | 0.74 |
| | | Logistic Regression | Model 2: AUC—0.84, |
| | | | Sensitivity—0.78, Specificity— |
| | | | 0.78 |
| Clinical Outcome | Markers | Machine Learning Models | Performance Metrics |
| Heterotopic Ossification in | IL-8, IL-2R, MIP-1a, GM-CSF, IL-15, IL- | Boruta, Random Forest | AUC—0.82, Sensitivity, 0.88, |
| Combat Related Extremity | 12, G-CSF, MIP-1b, IL-1 RA, RANTES, | | Specificity—0.61 |
| Trauma | wound surface area | | |
| Heterotopic Ossification from | Wound surface area, serum IL8, and | Classification and | AUC—0.83, Sensitivity— |
| High-Energy Penetrating | effluent IL7 | Regression Tree | 0.80, Specificity—0.90 |
| Traumas | | | |
| Pneumonia | ISS, AIS chest, cryoprecipitate, FGF- | Backwards Elimination, | AUC—0.95 |
| | basic, IL-2R, and IL-6 | Random Forest, Logistic | |
| | | Regression, Leave one out | |
| | | cross validation | |
| Post-traumatic Sterile | mtDNA and SCD40L | | R² = 0.18323, p = 0.042 |
| inflammation | | | |
| Sepsis | APACHE GCS verbal score, injury GCS | Backwards Elimination, | AUC—0.90, Sensitivity— |
| Vasospasm and Mortality | verbal score, 2 penetrating abdominal | Random Forest | 0.88, Specificity—0.77 |
| Following Severe Traumatic | trauma index variables, serum FGF- | Backwards Elimination and | Vasospasm: AUC—6.87, |
| Brain Injury | basic, and serum IL-6, IL-8 and VEGF | Random Forest | Sensitivity—0.85, Specificity— |
| | Marshall Classification 2 (mild diffuse | | 0.81 |
| | injury), presence of midline shift based | | Mortality—0.90, Sensitivity— |
| | on Rott CT, and temperature | | 0.86, Specificity—0.87 |
| Wound Closure | Serum FGFb, IL-12, EGF, IL-10, IL-1β, | Boruta, Random Forest | AUC—0.81, Sensitivity— |
| | VEGF and effluent G-CSF, IL-17, TNF-α, | | 0.70, Specificity—0.40 |

The clinical parameters may include one or more biological effectors and/or one or more non-biological effectors. As used herein, the term “biological effector” or “biomarker” is used to mean a molecule, such as but not limited to, a protein, peptide, a carbohydrate, a fatty acid, a nucleic acid, a glycoprotein, a proteoglycan, etc. that can be assayed. Specific examples of biological effectors can include, cytokines, growth factors, antibodies, hormones, cell surface receptors, cell surface proteins, carbohydrates, etc. More specific examples of biological effectors include interleukins (ILs) such as IL-1α, IL-1β, IL-1 receptor antagonist (IL-1RA), IL-2, IL-2 receptor (IL-2R), IL-3, IL-4, IL-5, IL-6, IL-7, IL-8, IL-10, IL-12, IL-13, IL-15, IL-17, as well as growth factors such as tumor necrosis factor alpha (TNFα), granulocyte colony stimulating factor (G-CSF), granulocyte macrophage colony stimulating factor (GM-CSF), interferon alpha (IFN-α), interferon gamma (IFN-γ), epithelial growth factor (EGF), basic endothelial growth factor (bEGF), hepatocyte growth factor (HGF), vascular endothelial growth factor (VEGF), and chemokines such as monocyte chemoattractant protein-1 (CCL2/MCP-1), macrophage inflammatory protein-1 alpha (CCL3/MIP-1α), macrophage inflammatory protein-1 beta (CCL4/MIP-1β), CCL5/RANTES, CCL11/eotaxin, monokine induced by gamma interferon (CXCL9/MIG) and interferon gamma-induced protein-10 (CXCL10/IP10). In embodiments, the biological effectors are soluble. In embodiments, the biological effectors are membrane-bound, such as a cell surface receptor. In embodiments, the biological effectors are detectable in a fluid sample of a subject such as serum, wound effluent, and/or plasma. In embodiments, the clinical parameters include one or more proteins, nucleic acids, cytokines, growth factors, and others.

As used herein, the term non-biological effector is a clinical parameter that is generally considered not to be a specific molecule. Although not a specific molecule, a non-biological effector may nonetheless still be quantifiable, either through routine measurements or through measurements that stratify the data being assessed. For example, number or concentrate of red blood cells, white blood cells, platelets, coagulation time, blood oxygen content, etc. would be a non-biological effector component of the risk profile. All of these components are measurable or quantifiable using routine methods and equipment. Other non-biological components include data that may not be readily or routinely quantifiable or that may require a practitioner's judgment or opinion. For example, wound severity may be a component of the risk profile. While there may be published guidance on classifying wound severity, stratifying wound severity and, for example, assigning a numerical value to the severity, still involves observation and, to a certain extent, judgment or opinion. In some instances, the quantity or measurement assigned to a non-biological effector could be binary, e.g., “0” if absent or “1” if present. In other instances, the non-biological effector aspect of the risk profile may involve qualitative components that cannot or should not be quantified.

In embodiments, the mechanism of injury is a clinical parameter. As used herein, the phrase “mechanism of injury” means the manner in which the subject received an injury and may fall into one of three categories: blast, crush, or gunshot wound (GSW). A blast injury is a complex type of physical trauma resulting from direct or indirect exposure to an explosion. Blast injuries may occur, for example, with the detonation of high-order explosives as well as the deflagration of low order explosives. Blast injuries may be compounded when the explosion occurs in a confined space. A crush injury is injury by an object that causes compression of the body. Crush injuries are common following a natural disaster or after some form of trauma from a deliberate attack. A GSW is an injury that occurs when a subject is shot by a bullet or other sort of projectile from a firearm. Abdominal Trauma Index Scores are scores that are based on several different methods of assessing the level of trauma that occurs to the abdominal region. For examples, Abdominal Trauma Index Scores may comprise one or more of, but is not limited to the Abdominal Trauma Index, the Penetrating Abdominal Trauma Index, the Injury Severity Score. The Injury GCS Score Verbal is the subset of the GCS score wherein verbal response is scored based on the following criteria: oriented (5 points), confused conversation but able to answer questions (4 points), inappropriate word choice (3 points), incomprehensible speech (2 points), no response (1 point). The Sequential Organ Failure Assessment (SOFA) score measures the number and severity of organ dysfunction in six organ systems (respiratory, coagulatory, liver, cardiovascular, renal, and neurological). As such, the SOFA score can measure individual or aggregate organ dysfunction. Other markers of injury are important clinical parameters, including, but not limited to, location of injury, presence of abdominal injury, mechanism of injury, wound depth, wound surface area, number of wound debridements, associated injuries, type of wound closure, success of wound closure, wound presence and location, compound fracture, soft tissue injury, and limb amputation

Levels of the clinical parameters can be assayed, detected, measured, and/or determined in a sample taken or isolated from a subject. “Sample” and “test sample” are used interchangeably herein.

Examples of test samples or sources of clinical parameters include, but are not limited to, biological fluids and/or tissues isolated from a subject or patient, which can be tested by the methods of the present application described herein, and include but are not limited to whole blood, peripheral blood, serum, plasma, cerebrospinal fluid, wound effluent, urine, amniotic fluid, peritoneal fluid, pleural fluid, lymph fluids, various external secretions of the respiratory, intestinal, and genitourinary tracts, tears, saliva, white blood cells, solid tumors, lymphomas, leukemias, myelomas, and combinations thereof. In particular embodiments, the sample is a serum sample, wound effluent, or a plasma sample.

In embodiments, the clinical parameters are one or more of biomarkers, administration of blood transfusion products (blood products), administration of cryoprecipitate, and injury severity scores. In embodiments, the clinical parameters of a subject are selected from one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, or 45 of the clinical parameters mentioned here in.

In embodiments of the methods disclosed herein, the clinical parameters are selected from one or more of units of total blood products transfused in the first 24 hours, soft tissue injury, level of interferon interleukin-15 (IL-15) in a serum sample from the subject, level of Monokine induced by gamma interferon (MIG) in a serum sample from the subject, and level of vascular endothelial growth factor (VEGF) in a serum sample from the subject.

As used herein, the term “summation of all blood products administered to the subject” refers to a value reflecting the total amount of blood products administered to the subject. Blood products include but are not limited to whole blood, platelets, red blood cells, packed red blood cells, and serum. In embodiments, this value reflects the total amount of blood products needed to stabilize the subject following hemorrhage. Blood products may be delivered in values of 1 unit or more, 10 units or more, 20 units or more, 30 units or more, 40 units or more, 50 units or more, 100 units or more, 150 units or more, 200 units or more, 250 units or more, 300 units or more, 350 units or more, 400 units or more, 450 units or more, or 500 units or more. Stabilization refers to homeostasis achieved in the subject and is defined as either achieving an equilibrium between bleeding or a complete cessation of hemorrhage in the subject.

In embodiments, administration of coagulant data is included. Administration of anticoagulants may include at least one of tranexamic acid administration (TXA), epsilon-aminocaproic acid, or aminomethylbenzoic acid.

As used herein, the term “AIS” refers to the abbreviated injury scale, a well-known parameter in the art used routinely in clinics to assess severity of wounds or injuries. In embodiments, an AIS of 1 is a minor injury, an AIS of 2 is a moderate injury, and AIS of 3 is a serious injury, an AIS of 4 is a severe injury, an AIS of 5 is a critical injury, and an AIS of 6 is a nonsurvivable injury.

In embodiments, injury data includes injury location data, type of injury data, and injury size data. Injury location data includes at least one of upper extremity injury wound, lower extremity wound, lateral thigh wound, posterior thigh wound, calf wound, and forearm wound. Injury type data includes at least one of open fracture, amputation, soft tissue injury, or wound. Injury size data includes at least one of volume of wound or surface area of wound.

As used herein, the term critical colonization (or “CC”) is a measure of CFU that the subject has in serum and/or tissue for at least one wound when initially examined by the attending physician. For example, if a subject has CFU of 1×10⁵ per ml of serum, or if at least one wound has CFU of 1×10⁵ per mg of tissue, the subject is said to be “positive” for CC. If the total serum CFU or no single wound has CFU of at least 1×10⁵ the subject is said to be “negative” for CC.

As used herein, assessing an injury such as an abdominal injury and/or a head injury, for the purposes of using these clinical parameters in the systems and methods described herein, means determining the degree or extent of injury, as reflected in an AIS score of 1-6.

In various embodiments, systems, methods, and a non-transitory computer-readable medium of the present disclosure can execute a process by which data is aggregated about one or more subjects, machine learning algorithms perform data-mining procedures, pattern recognition, intelligent prediction, and other artificial intelligence procedures, such as for enabling diagnostic predictions based on clinical parameters. Machine learning algorithms are increasingly being implemented to reveal knowledge structures that guide decisions in conditions of limited certainty which can lead to improved decision making. Using manual techniques or traditional algorithmic approaches, this would not be possible because of the large number of data points involved. However, in order to use machine learning algorithms effectively, a machine learning engine comprising a specific sequence of approaches and comparisons of models implemented by machine learning algorithms may be required in order to get optimal results out of existing data.

Constructing such a machine learning engine and executing these machine learning algorithms can improve the performance of diagnostic prediction technology. These improvements may include, but are not limited to by increasing accuracy, selectivity, and/or specificity of models used to perform the diagnostic predictions. Therefore, such an engine can improve decision-making for and delivery of treatments to subjects. While various machine learning algorithms can be used for such purposes, generating a machine learning engine with desired performance characteristics can be highly domain-specific, requiring rigorous modeling, testing, and validation to select appropriate algorithms (or combinations thereof) and the parameters modeled with the algorithms to generate the machine learning system.

In embodiments, the machine learning engine may be constructed to include five major components: (1) initial data exploration, (2) feature selection, (3) prediction, (4) model selection via validation, (5) deployment and self-improvement. It will be understood by those possessing ordinary skill in the art that these stages may not be discrete entities and there may be overlap between them, and that the output from each stage may be used to inform, calibrate, and/or improve other stages of the engine and the machine learning engine.

The initial data stage may include data preparation. Data preparation may include cleaning data (e.g., searching for outlying data, applying missing data algorithms, alter data formats), transforming data, selecting subsets of records and—in case of data sets with large numbers of variables (“fields or dimensions”). The data on which data preparation is performed may be referred to as training data.

In embodiments, data preparation can include executing pre-processing operations on the data. For example, missing data may be handled through the execution of imputation algorithms that interpolate and/or estimate missing values. One example of imputation involves generating a distribution (e.g. Gaussian, Poisson, binomial, zero-inflation, beta, pert) of available data for a clinical parameter having missing data, and interpolating values for the missing data based on the distribution.

The feature selection stage involves performing feature selection operations (e.g., feature selection, parameter selection), to bring the number of variables to a manageable and appropriate range (depending on the statistical methods which are being considered). In many supervised learning problems, feature selection can be important for a variety of reasons including generalization performance, running time requirements and constraints and interpretational issues imposed by the problem itself (e.g., understanding direction of causality, removing known confounders). The data on which the feature selection is performed may be referred to as training data. Given that the performance of machine learning algorithms can depend strongly on the quality of the training data used to train the algorithms, feature selection and other data preparation operations can be highly significant for ensuring desired performance.

In embodiments, feature selection can include executing supervised machine learning algorithms, such as constraint-based algorithms, constrain-based structure learning algorithms, and/or constraint-based local discovery learning algorithms. Feature selection can be executed to identify a subset of variables in the training data which have desired predictive ability relative to a remainder of the variables in the training data, enabling more efficient and accurate predictions using a model generated based on the selected variables. In embodiments, feature selection is performed using machine learning algorithms from the “bnlearn” R package, including but not limited to the Grow-Shrink (“gs”), Incremental Association Markov Blanket (“iamb”), Fast Incremental Association (“fast.iamb”), Max-Min Parents & Children (“mmpc”),Semi-Interleaved Hiton-PC (“si.hiton.pc”), or Backwards Variable Elimination algorithms. R is a programming language and software environment for statistical computing. It will be appreciated that various other implementations of such machine learning algorithms (in R or other environments such as Python) may be used to perform feature selection and other processes described herein. Feature selection can search for a smaller dimension set of variables that seek to represent the underlying distribution of the full set of variables, which attempts to increase generalizability to other data sets from the same distribution.

In embodiments, feature selection is performed to search the training data for a subset of variables using Backwards Variable Elimination. Backwards Elimination begins with all the variables and removes variables in a backwards, stepwise manner. At each step the model is tested to see if it is improved by the removal of a variable. The removal of variables ends when removing variables no longer improves the model and the optimal selection of variables is achieved. Exemplary embodiments of feature selection machine learning models that have proven to improve prediction based on the present markers and training data are listed by clinical outcome in Table 1.

The prediction stage involves the task of generalizing a known structure and applying it to new data. Prediction algorithms can include, but are not limited to linear discriminant analysis, classification and regression trees/decision tree learning/random forest modeling, nearest neighbor, support vector machine, logistic regression, generalized linear models, Naive Bayesian classification, and neural networks, among others. In embodiments, prediction algorithms can be used from the train function of the R caret package, including but not limited to linear discriminant analysis (lda), classification and regression trees (cart), k-nearest neighbors (knn), support vector machine (svm), logistic regression (glm), random forest (rf), generalized linear models (glmnet) and/or naïve Bayes (nb). These prediction algorithms can also be referred to as classification algorithms and are used to form predictions of the presence (i.e., is the clinical outcome present or not) or development (i.e., will the clinical outcome develop or not) of a clinical outcome. While these prediction algorithms are disclosed, many others are contemplated, including k-means cluster, non-linear clustering, boosted trees, mixture models, and/or OPTICS algorithms.

For example, in one variant of a machine learning model a random forest mode may be utilized. A random forest can include a “forest” of a large number of decision trees, such as on the order of 10² to 10⁵ decision trees. The number of decision trees may be selected by calculating an OOB error (the mean prediction error on each training sample, using only the trees that did not have each training sample in their randomly sampled set of training data, as discussed below) for the resulting random forest model. In embodiments, the number of decision trees used may be several hundred trees, which can improve computational performance of the machine learning systems by reducing the number of calculations needed to execute the random forest model. The two chief draws of the random forest are that it does not require the data to be either normally distribution or transformed and that the algorithm requires little tuning, which is advantageous when updating data sets, and its numerical process includes cross validation precluding the need for post model-building cross validation.

In embodiments, each random forest decision tree is generated by bootstrap aggregating (“bagging”), where for each decision tree, the training data is randomly sampled with replacement to generate a randomly sampled set of training data, and then the decision tree is trained on the randomly sampled set of training data. In embodiments, where feature selection is performed prior to generated the random forest model, the training data is sampled based on the reduced set of variables from feature selection (as opposed to sampling based on all variables).

To perform a prediction given values of variables for a subject, each decision tree is traversed using the given values until a decision rule is reached that is followed by terminal nodes (e.g., presence of disease in the subject, no presence of disease in the subject). The outcome from the decision rule followed by the terminal nodes is then used as the outcome for the decision tree. The outcomes across all decision trees in the random forest model are summed to generate a prediction regarding the subject.

In a separate variant of a prediction machine learning model, a neural network includes a plurality of layers each including one or more nodes, such as a first layer (e.g., an input layer), a second layer (e.g., an output layer), and one or more hidden layers. The neural network can include characteristics such weights and biases associated with computations that can be performed between nodes of layers. For example, a node of the input layer can receive input data, perform a computation on the input data, and output a result of the computation to a hidden layer. The hidden layer may receive outputs from one or more input layer nodes, perform a computation on the received output(s), and output a result to another hidden layer, or to the output layer. The weights and biases can affect the computations performed by each node and can be manipulated by an algorithm executing the neural network, such as an optimization algorithm being used to train the neural network to match training data. Neural networks describe a generalized approach to prediction and many different variants of neural networks may be used, including, but not limited to: convolutional neural networks, deep belief networks, deep reservoir computing, restricted Boltzmann machines, deep stacking networks, tensor deep stacking networks, and/or hierarchical-deep models.

In another variant of a machine learning model, Naïve Bayesian algorithms can apply Bayes' theorem to predict outcomes based on values of variables, such as values of the variables identified using feature selection. The model is called “naïve” due to the assumption that each of the variables is independently associated with having venous thromboembolism. While it may be more realistic for there to be a joint probability for the variables when performing predictions, the naïve approach may provide performance characteristics desirable for the diagnostic prediction system being generated. A naïve Bayes model can be trained by calculating a relationship between values of each variable and the corresponding outcome(s) represented in the training data. For example, in a diagnostic prediction system for a clinical outcome, values of each variable may be associated with the outcomes of whether or not the particular clinical outcome is present. In embodiments, the relationship may be calculated using a normal distribution for the values of the variables, such that the normal distribution can be used to determine a probability that each variable may have a specified value in the case of (1) the clinical outcome being present, or (2) the clinical outcome not being present. Then, when executing the trained naïve Bayes to predict the presence of the particular disease for a subject, a probability can be calculated, for each value of each variable, that the variable would have that value given that the particular disease is present in the subject; similarly, a probability can be calculated, for each value of each variable, that the variable would have that value given that the clinical outcome is not present in the subject. The probabilities for each case can be combined, and then compared to generate a prediction as to whether the clinical outcome is present in the subject.

Regression analysis attempts to find a function which models the data with the least error. Regression analysis can be used for prediction, as the function can be used to predict a value for a dependent variable given value(s) for independent variable(s). In some examples, logistic regression can be used to classify the presence or absence of a clinical outcome. Regressions can involve linear regressions, which involves a linear approach to modeling the relationship between a dependent variable and one or more independent variables, or they can involve non-linear regressions in which are regression in which the dependent or criterion variables are modeled as a non-linear function of model parameters and one or more independent variables.

Model selection includes but is not limited to considering the various machine learning algorithms and models and selecting the best one based on predictive performance. Predictive performance can correspond to explaining the variability in question and producing stable results across samples. This process requires a systematic and elaborate approach to screening a multitude of models across various metrics and comparing results across models, outcomes, and populations. There are a variety of techniques developed to achieve that goal—many of which are based on so-called “competitive evaluation of models”, that is, applying different models to the same data set and then comparing their performance to choose the best model. These techniques—which are often considered the core of predictive machine learning—can include: bagging (voting, averaging), boosting, stacking (stacked generalizations), and meta-learning. Validation can include comparing the output of a selected model to validation data. For example, a portion of the training data can be held separately from that which is used to train the model, and then can be used to confirm the performance characteristics of the trained model. The portion of training data that can be held separately from that which is used to train the model can include 0.1-1% of the data, 1-5% of the data, 1-10% of the data, 1-15% of the data, 1-20% of the data, 1-25% of the data, 1-30% of the data, 1-35% of the data, 1-40% of the data, 1-45% of the data, 1-50% of the data, 1-55% of the data, 1-60% of the data, 1-75% of the data, 1-80% of the data, 1-85% of the data, 1-90% of the data, 1-95% of the data, or 1-99% of the data.

In embodiments a comparison of machine learning algorithms (and combinations thereof) may be useful. Many application scenarios do not have single models, but multiple, related ones. Some typical examples are machine learning algorithms trained based on data derived at different points in time or in different subsets of the data, e.g., production quality data from different production sites. Another common case is representing the same data with machine learning algorithms on different types of machine learning algorithms in order to capture different aspects of the data. In all these cases, not only the individual data mining models are of interest, but also similarities and differences between them. Such differences may tell, for instance, how production quality and dependencies develop over time, how machine learning algorithms of different types differ in their ways of representing different products produced at the same facility or, how the production facilities differ between each other. Current examples of prediction machine learning that have proven to improve prediction based on the present markers and training data are listed by clinical outcome in Table 1. While the models in Table 1 reflect the most current understanding and application of machine learning models, as the machine learning engine and prediction engine is trained on more data and updated models, the markers and machine learning models that best fit each clinical outcome may change.

Machine learning algorithms (and combinations thereof) can be compared using performance metrics. The performance metrics may be selected based on the intended application of the machine learning algorithms (and the predictive models created using the machine learning algorithms). For diagnostic prediction models, the performance metrics can include but is not limited to Kappa score, Accuracy score, sensitivity, specificity, total, positive class, and negative class out-of-bag (OOB) error estimates, receiver operator characteristic curves (ROCs), areas under curve (AUCs), confusion matrices, Vickers and Elkins' Decision Curve Analysis (DCA), or other measures of the performance of the machine learning algorithms

The Kappa score represents a comparison of an observed accuracy of the diagnostic prediction model to an expected accuracy. For example, the Kappa score measures how closely the diagnostic prediction model matches training data (e.g., the relationships between variables and the corresponding outcomes known in the training data), controlling for the accuracy of a random classifier as measured by the expected accuracy.

The sensitivity measures a proportion of positive results from the prediction that are correctly identified as such. As such, the sensitivity can quantify the avoidance of false negatives. The specificity measures a proportion of negative results from the prediction that are correctly identified as such.

OOB measures prediction error in random forest and other machine learning techniques that rely on bootstrapping to sub-sample training data. The OOB error analysis can be used to show how the variable selected models can improve the OOB error (predictive performance) for the positive class.

The ROC curve is a plot of true positive rate (sensitivity) as a function of false positive rate (specificity). The AUC represents the area under the ROC curve, wherein a 1 represents a perfect model fit with 100% sensitivity and specificity. For example, model performance can be further assessed using the plot.roc command in R to compute the Receiver Operator Characteristic Curves (ROC) and area under curve (AUC).

Decision Curve Analysis (DCA) can be used to calculate the net benefit of treatment based on the diagnoses predicted by the diagnostic prediction models, as compared to baseline treatment methodologies such as assuming that all patients are test positive and therefore treating everyone, or assuming that all patients are test negative and therefore offering treatment to no one. The DCA curve plots the net benefit of the diagnostic prediction model as a function of a threshold probability, the threshold probability being a value at which the subject would opt for treatment given the relative harms of false positive predictions. DCA is used to compare various predictive and diagnostic paradigms in terms of net benefit to the patient. A typical DCA analysis will compare the null model, treat no one, to various alternative models, such as “treat-all” or treat according to the guidance of models built on biomarker predictors. DCA analysis can be interpreted as showing positive net-benefit to the patient if the decision curve for a particular model is above the null model (x axis), and to the right of the “treat-all” model. Net-benefit is defined mathematically as a summation of model performance (for instance propensity to predict false positive or false negatives) over a series of predictive threshold cutoffs and the respective sensitivity/specificity at those thresholds. The threshold cutoffs could be thought of as the point at which a decision to treat would be made given the relative harms and benefits of treating given the uncertainty of the prediction at that threshold. This analysis demonstrates the threshold cutoffs where the predictive models are most useful to the patient. The dca R command from the Memorial Sloan Kettering Cancer Center website, www.mskcc.org, can be used to compute the Decision Curve Analysis (DCA).

Deployment and self-improvement can include using the model selected as the best model in the previous stage and deploying it to a prediction engine. This prediction engine takes in new data, applies the selected model, and generates predictions or estimations of the expected venous thromboembolism. For example, the selected model can be executed using clinical parameters specific to a particular subject in order to predict the expected outcome for the particular subject. In embodiments, the clinical parameters for the particular subject can be used to update the machine learning engine, particularly after confirming whether or not the disease is present in the particular subject. As such, the machine learning engine adopts an iterative, self-improvement process upon deployment.

In embodiments, the systems and methods described herein for generating predictive models for predicting subject-specific venous thromboembolism outcomes involve the execution of two main steps: feature selection and multi-class prediction. An advantage of feature selection is that feature selection can search for a smaller dimension set of variables that seek to represent the underlying distribution of the full set of variables, which attempts to increase generalizability to other data sets from the same distribution. In embodiments, such as where the datasets are relatively small, computational time may not be a consideration. Since feature selection is based on a better representation of the underlying distribution of the full variables set, in theory, they should be more generalizable and less susceptible to over fitting.

In building machine learning engines to predict venous thromboembolism, it is typically unfeasible and unwarranted to provide the machine learning algorithms with an exhaustive list of clinical parameters which may be relevant to the venous thromboembolism being predicted. For example, with very large lists of clinical parameters, there may be significant noise, multicollinearity, large amounts of missing data, and other opportunities for introducing errors which can adversely affect the ability of the machine learning algorithms to generate predictive models (by performing feature selection and prediction) which meet desired performance metrics.

In some situations, the number of clinical parameters may be on the order of 5000-50000 variables, from which the machine learning solutions will have to perform feature selection and other operations. To do so would require incredibly large computing resources which are not readily available, making such processes virtually impossible. Additionally, even if such resources were available, the opportunities for introducing error in the resulting solutions would counteract any added benefit from considering all variables.

In embodiments, over 7000 initial clinical and nonclinical parameters are available regarding the subjects that could potentially be used to train the machine learning solutions. These clinical parameters fall into a wide variety of categories, such as demographics, wound type, wound mechanism, wound location, fracture characteristics, administration of blood products, injury severity scores, treatment(s), tobacco usage, activity levels, surgical history, nutrition, serum protein expression, wound effluent protein expression, tissue bacteriology, mRNA expression, and Raman spectroscopy. In embodiments, these categories, using expert knowledge, the following are selected for usage with the machine learning solutions disclosed herein: serum protein expression, administration of blood products, and injury severity scores. The expert selection process is based upon many criteria, including, but not limited to established causal mechanisms of action for disease processes or venous thromboembolism, diagnostic algorithms. The expert selection process is important for distilling the many possible parameters to the minimum number that will result in the strongest predictive power.

In embodiments, clinical parameters that fall within the serum protein expression include but are not limited to any one or more of level of interleukin-1α (IL-1α) in a sample from the subject, level of interleukin-Iβ (IL-Iβ) in a sample from the subject, level of interleukin-1 receptor agonist (IL-1RA) in a sample from the subject, level of interleukin-2 (IL-2) in a sample from the subject, level of interleukin-2 receptor (IL-2R) in a sample from the subject, level of interleukin-3 (IL-3) in a sample from the subject, level of interleukin-4 (IL-4) in a sample from the subject, level of interleukin-5 (IL-5) in a sample from the subject, level of interleukin-6 (IL-6) in a sample from the subject, level of interleukin-7 (IL-7) in a sample from the subject, level of interleukin-8 (IL-8) in a sample from the subject, level of interleukin-10 (IL-10) in a sample from the subject, level of interleukin-12 (IL-12) in a sample from the subject, level of interleukin-13 (IL-13) in a sample from the subject, level of interleukin-15 (IL-15) in a sample from the subject, level of interleukin-17 (IL-17) in a sample from the subject, level of tumor necrosis factor alpha (TNF-α) in a sample from the subject, level of granulocyte colony stimulating factor (G-CSF) in a sample from the subject, level of granulocyte macrophage colony stimulating factor (GM-CSF) in a sample from the subject, level of interferon alpha (IFN-α) in a sample from the subject, level of interferon gamma (IFN-γ) in a sample from the subject, level of epithelial growth factor (EGF) in a sample from the subject, level of hepatocyte growth factor (HGF) in a sample from the subject, the level of vascular endothelial growth factor (VEGF) in a sample from a subject, the level of monocyte chemoattractant protein-1 (CCL2/MCP-1) in a sample from a subject, the level of macrophage inflammatory protein-1 alpha (CCL3/MIP-Iα) in a sample from a subject, the level of macrophage inflammatory protein-1 beta (CCL4/MIP-Iβ) in a sample from a subject, the level of CCL5/RANTES in a sample from a subject, the level of CCL11/eotaxin in a sample from a subject, the level of monokine induced by gamma interferon (CXCL9/MIG) in a sample from a subject, the level of interferon gamma-induced protein-10 (CXCL10/I P10) in a sample from a subject, the level of basic fibroblast growth factor (bFGF) in a sample from a subject, the level of mitochondrial DNA in a sample from a subject, the level of soluble CD40 ligand (sCD40L) in a subject, the level of transglutaminase 2 in a sample from a subject, among others.

In embodiments, clinical parameters that fall within the administration of blood products category include amount of whole blood cells administered to the subject, units of total blood products transfused in the first 24 hours, amount of red blood cells (RBCs) administered to the subject, amount of packed red blood cells (pRBCs) administered to the subject, amount of platelets administered to the subject, summation of all blood products administered to the subject, and/or level of total packed RBCs, among others.

In embodiments, clinical parameters that fall within the injury severity scores category include Injury Severity Score (ISS), Abbreviated Injury Scale (AIS) of abdomen, AIS of chest (thorax), AIS of extremity, AIS of face, AIS of head, and/or AIS of skin, among others.

In embodiments, subject data includes at least one of gender, age, date of injury, length of hospital stay, length of intensive care unit (ICU) stay, number of days on a ventilator, disposition from hospital, development of nosocomial infections, sequential organ failure assessment (SOFA), injury GCS score, Marshall Classification 2 (mild diffuse injury), midline shift based on Roterdam computed tomography, temperature, arterial pH, potassium score, vascular injury score, pulse rate, or FiO₂.

The machine learning solutions described herein can execute feature selection on the clinical parameters within the identified categories to generate predictive models for predicting venous thromboembolism.

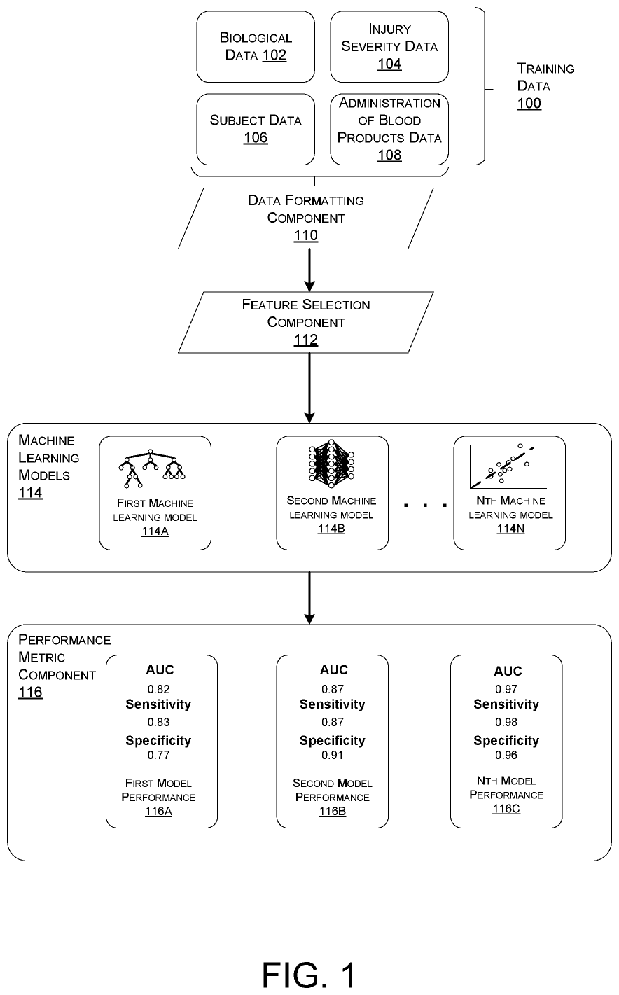

Referring now to FIG. 1, the process of an embodiment and its components are shown according to an embodiment of the present disclosure. The machine learning engine starts with training data 100 and executes data formatting in a data formatting component 110. The feature selection occurs in a feature selection component 112, then the formatted data is fed into machine learning models 114 wherein prediction of venous thromboembolism occurs.

In embodiments, the training data 100 comprises clinical parameters including biological data 102, injury severity data 104, subject data 106, and administration of blood products data 108.

In embodiments, the injury severity data 104 may include, but is not limited to one or more of Injury Severity Score (ISS), Abbreviated injury scale (AIS) of abdomen, AIS of chest (thorax), AIS of extremity, AIS of face, AIS of head, or AIS of skin, location of injury, presence of abdominal injury, mechanism of injury, wound depth, wound surface area, number of wound debridements, associated injuries, type of wound closure, success of wound closure, wound presence and location, compound fracture, soft tissue injury, and limb amputation. The injury severity data may also include data on mechanism of injury, such as: blast wound, crush wound, gunshot wound, impalement wound. A blast injury is a complex type of physical trauma resulting from direct or indirect exposure to an explosion. Blast injuries may occur, for example, with the detonation of high-order explosives as well as the deflagration of low order explosives. Blast injuries may be compounded when the explosion occurs in a confined space. A crush injury is injury by an object that causes compression of the body. Crush injuries are common following a natural disaster or after some form of trauma from a deliberate attack. A gunshot wound is an injury that occurs when a subject is shot by a bullet or other sort of projectile from a firearm. An impalement wound is an injury involving pierced or transfixed with a sharp object.

In embodiments, the biological data 102 may include, but is not limited to one or more of interleukin-1α (IL-1α) in a sample from the subject, level of interleukin-Iβ (IL-Iβ) in a sample from the subject, level of interleukin-1 receptor agonist (IL-1RA) in a sample from the subject, level of interleukin-2 (IL-2) in a sample from the subject, level of interleukin-2 receptor (IL-2R) in a sample from the subject, level of interleukin-3 (IL-3) in a sample from the subject, level of interleukin-4 (IL-4) in a sample from the subject, level of interleukin-5 (IL-5) in a sample from the subject, level of interleukin-6 (IL-6) in a sample from the subject, level of interleukin-7 (IL-7) in a sample from the subject, level of interleukin-8 (IL-8) in a sample from the subject, level of interleukin-10 (IL-10) in a sample from the subject, level of interleukin-12 (IL-12) in a sample from the subject, level of interleukin-13 (IL-13) in a sample from the subject, level of interleukin-15 (IL-15) in a sample from the subject, level of interleukin-17 (IL-17) in a sample from the subject, level of tumor necrosis factor alpha (TNF-α) in a sample from the subject, level of granulocyte colony stimulating factor (G-CSF) in a sample from the subject, level of granulocyte macrophage colony stimulating factor (GM-CSF) in a sample from the subject, level of interferon alpha (IFN-α) in a sample from the subject, level of interleukin-8 (IL-8) in a sample from the subject, level of interleukin-10 (IL-10) in a sample from the subject, level of interferon gamma (IFN-γ) in a sample from the subject, level of epithelial growth factor (EGF) in a sample from the subject, level of basic epithelial growth factor (bFGF) in a sample from the subject, level of hepatocyte growth factor (HGF) in a sample from the subject, the level of vascular endothelial growth factor (VEGF) in a sample from a subject, the level of monocyte chemoattractant protein-1 (CCL2/MCP-1) in a sample from a subject, the level of macrophage inflammatory protein-1 alpha (CCL3/MIP-Ia) in a sample from a subject, the level of macrophage inflammatory protein-1 beta (CCL4/M I P-Iβ) in a sample from a subject, the level of CCL5/RANTES in a sample from a subject, the level of CCL11/eotaxin in a sample from a subject, the level of monokine induced by gamma interferon (CXCL9/MIG) in a sample from a subject, the level of interferon gamma-induced protein-10 (CXCL10/I P10) in a sample from a subject, the level of basic fibroblast growth factor (bFGF) in a sample from a subject, the level of mitochondrial DNA in a sample from a subject, the level of soluble CD40 ligand (sCD40L) in a subject, the level of transglutaminase 2 in a sample from a subject, gender, age, date of injury, location of injury, presence of abdominal injury, mechanism of injury, wound depth, wound surface area, number of wound debridements, associated injuries, type of wound closure, success of wound closure, requirement for transfusion, total number of blood products transfused, units of total blood products transfused in the first 24 hours, amount of whole blood cells administered to the subject, amount of red blood cells (RBCs) administered to the subject, amount of packed red blood cells (pRBCs) administered to the subject, amount of platelets administered to the subject, level of total packed RBCs, Injury Severity Score (ISS), Abbreviated Injury Scale (AIS) of head, AIS of abdomen, AIS of chest (thorax), Acute Physiology and Chronic Health Evaluation II (APACHE II) score, presence of critical colonization (CC) in a sample from the subject, presence of traumatic brain injury, severity of traumatic brain injury, length of hospital stay, length of intensive care unit (ICU) stay, number of days on a ventilator, disposition from hospital, development of nosocomial infections, sequential organ failure assessment, injury GCS score, Marshall Classification 2 (mild diffuse injury), midline shift based on Rotterdam computed tomography temperature, arterial pH, pulse rate, and FiO₂.

In embodiments, the subject data 106 comprise one or more of: gender, age, date of injury, length of hospital stay, length of intensive care unit (ICU) stay, number of days on a ventilator, disposition from hospital, development of nosocomial infections, sequential organ failure assessment (SOFA), injury GCS score, Marshall Classification 2 (mild diffuse injury), midline shift based on Roterdam computed tomography, temperature, arterial pH, potassium score, vascular injury score, pulse rate, or FiO₂. While these subject data 106 are enumerated, many others are contemplated.

In embodiments, the administration of blood products data comprise one or more of: an amount of whole blood cells administered to the subject, amount of red blood cells (RBCs) administered to the subject, amount of packed red blood cells (PRBCs) administered to the subject, amount of platelets administered to the subject, units of blood products transfused in the first 24 hours, summation of all blood transfusion products administered to the subject, or a level of total packed RBCs. While these administration of blood products data 106 are enumerated, many others are contemplated.

In embodiments, the feature selection component 110 executes a series of feature selection algorithms. The feature selection algorithms include but are not limited to one or more of: backwards variable elimination algorithms, constraint-based algorithms, constraint-based structure learning algorithms, and/or constraint-based local discovery learning algorithms. For example, the machine learning engine 204 can execute machine learning algorithms from the “bnlearn” R package, including but not limited to the Grow-Shrink (“gs”), Incremental Association Markov Blanket (“iamb”), Fast Incremental Association (“fast.iamb”), Max-Min Parents & Children (“mmpc”), or Semi-Interleaved Hiton-PC (“si.hiton.pc”) algorithms. While these feature selection algorithms are enumerated, many others are contemplated. In embodiments, the feature selection results of the feature selection algorithms are compared, and the top performing variables are selected.

The machine learning models 114, are used to predict venous thromboembolism based on the clinical parameters selected during feature selection. Prediction machine learning models can include, but are not limited to random forest, linear discriminant analysis, classification and regression trees/decision tree learning/random forest modeling, nearest neighbor, support vector machine, logistic regression, generalized linear models, naive Bayesian classification, and neural networks, among others. In embodiments, prediction algorithms can be used from the train function of the R caret package, including but not limited to linear discriminant analysis (lda), classification and regression trees (cart), k-nearest neighbors (knn), support vector machine (svm), logistic regression (glm), random forest (rf), generalized linear models (glmnet) and/or naïve Bayes (nb). These algorithms are used to form predictions of the presence (i.e., is the clinical outcome present or not) or development (i.e., will the clinical outcome develop or not) of a clinical outcome. While these prediction algorithms are disclosed, many others are contemplated, including k-means cluster, non-linear clustering, boosted trees, mixture models, and/or OPTICS algorithms. The machine learning models 114 may be executed by identifying first values of clinical parameters in the training data 100 corresponding to each subset of model parameters and generating predictions of venous thromboembolism using the identified first values.

The machine learning engine 204 can use the predictions of venous thromboembolism to calculate performance metrics that are derived from the prediction results 132. For example, the machine learning engine 204 can calculate a performance metric for each combination of (i) a subset of model parameters (selected by each feature selection algorithm of the feature selection component 112) and (ii) a machine learning model 114 used to generate the predictions of venous thromboembolism. The performance metrics can represent the ability of each combination to predict venous thromboembolism.

The machine learning models 114 can calculate a performance metric including, but not limited to one or more an AUC, a sensitivity, or a specificity. In embodiments, the performance metric component 116 can generate a ROC curve based on the sensitivity and the specificity. The performance metric component 116 can also calculate an AUC based on the ROC curve. In embodiments, the machine learning model 114 can be evaluated by further performance metrics. For example, the machine learning model 114 can be evaluated based on Accuracy, No Information Rate, positive predictive value and negative predictive value.

The machine learning models 114 can apply various policies, heuristics, or other rules based on the performance metric(s) to select a machine learning model 114 for prediction (and corresponding subset of model parameters selected by one of the feature selection algorithms). For example, values for each performance metric can be compared to respective threshold values, and a machine learning model 114 can be determined to be a candidate machine learning model 114 (or a potential candidate) responsive to the value for the performance metric exceeding the threshold. The performance metric component 116 can assign weights to each performance metric to calculate a composite performance metric.

In embodiments, the prediction engine machine learning models 114 selects the candidate machine learning model 114 and corresponding subset of model parameters based on the rule: identify the combination having (1) a highest AUC score; subsequently, (2) a highest sensitivity; and (3) subsequently, a specificity greater than a threshold specificity.

The performance metric component can execute decision curve analysis (DCA) to evaluate the performance of the candidate machine learning model 114 and/or with confusion matrices. DCA can be used to assess the net benefit of using the candidate machine learning model 114 in a clinical setting as compared to a null model, a treat no one paradigm, or a “treat-all” intervention paradigm. The DCA can be executed to validate the performance of the candidate machine learning model 114, and/or to select the candidate machine learning model 114 from amongst several machine learning model 114having similar performance under other performance metrics.

The machine learning models can be executed in multiple iterations. For example, the data of the training database 100 can be run through the feature selection and machine learning models more than once, for example, 10, 20, 30, 40, 50 or even more times.

In embodiments, the candidate model (combination of subset of model parameters and candidate machine learning model 114) can be compared in performance to a model generated using the full set of clinical parameters of the training data 100. For example, a machine learning model 114 can be executed using the full set of clinical parameters, in a similar manner as for executing the machine learning model 114 based on the subsets of model parameters, to represent a baseline for model performance. The candidate model can be compared to the model generated using the full set of clinical parameters using DCA. The machine learning engine 204 can execute an imputation algorithm to process clinical parameters with missing data.

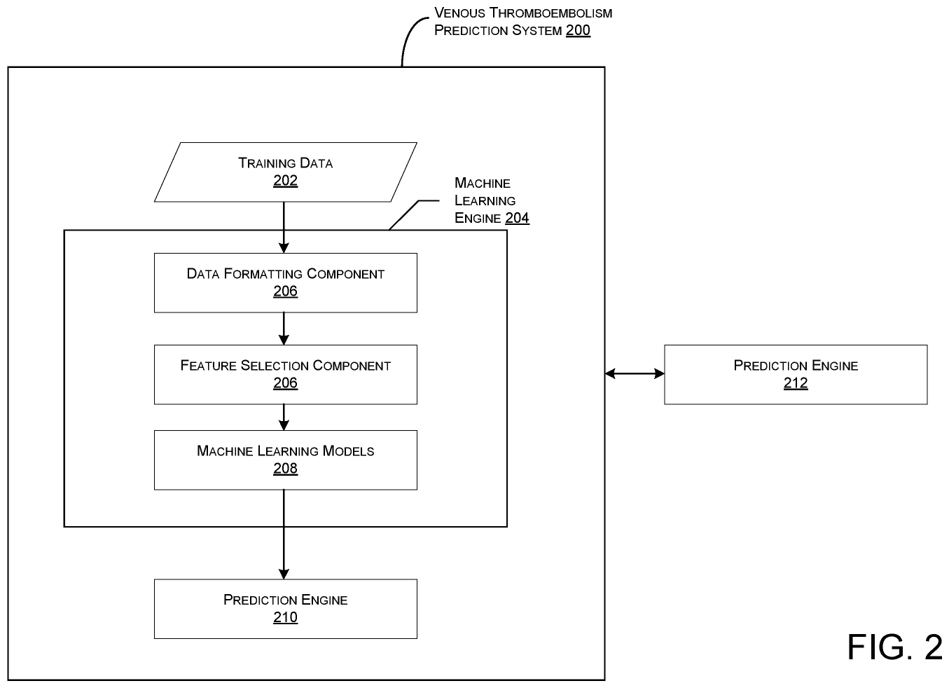

Referring to FIG. 2, in embodiments, the Venous Thromboembolism Prediction System 200 includes a prediction engine 210. The prediction engine 210 can predict venous thromboembolism specific to at least one second subject. The prediction engine 210 can receive, for the at least one second subject, a second value of at least one clinical parameter of the plurality of clinical parameters.

In embodiments, at least one of the received second values corresponds to a model parameter of the subset of model parameters used in the candidate machine learning model 114. If the prediction engine 210 receives several second values of clinical parameters, of which at least one does not correspond to a model parameter of the subset of model parameters, the prediction engine 210 may execute an imputation algorithm to generate a value for such a missing parameter.

The prediction engine 210 can execute the candidate machine learning model 114 using the corresponding subset of model parameters and the second value of the at least one clinical parameter to calculate the venous thromboembolism outcome specific to the at least one second subject. In an example, the candidate machine learning algorithm 114 may include a random forest model based on the following model parameters (and received the indicated second values for the second subject): IL-15 (200); MIG (25); VEGF (450); units of total blood products transfused in the first 24 hours (22); and soft tissue injury (6). Using these values, the prediction engine 210 can cause the candidate machine learning model 114 to calculate the probabilities that the second subject would have those values for the model parameters given that the subject has venous thromboembolism: IL-15 (0.85); MIG (0.92); VEGF (0.45); units of total blood products transfused in the first 24 hours (0.77); and soft tissue injury (0.92), resulting in an overall probability of 0.249. Similarly, the prediction engine 210 can determine an overall probability associated with the given not thromboembolism case to be approximately zero. As such, the prediction engine 210 can output a prediction that the second subject has venous thromboembolism based on the overall probabilities (e.g., based on a ratio of the overall probabilities).

As shown in FIG. 2, the venous thromboembolism prediction system 200 includes the prediction engine 210. In embodiments, a remote device 430 may additionally or alternatively include a separate, but similar prediction engine 212. The prediction engine 212 can incorporate features of the prediction engine 210. The remote device 430 can incorporate features of the computing environment. The remote device 430 can communicate with the venous thromboembolism prediction system 200 using any of a variety of wired or wireless communication protocols (including communicating via a network 428). For example, the remote device 430 can receive the prediction engine 210 (or the candidate machine learning model 114 with the corresponding subset of model parameters) from the venous thromboembolism prediction system 200.

In various embodiments, the venous thromboembolism prediction system 200 and/or the remote device 430 can receive the second values of the plurality of clinical parameters through a user interface and can output the predictions of venous thromboembolism responsive to receiving the second values. The remote device 430 can be implemented as a client device executing the prediction engine 212 as a local application which receives the second values and transmits the second values to the venous thromboembolism prediction system 200; the venous thromboembolism prediction system 200 can be implemented as a server device which calculates the prediction of the venous thromboembolism specific to the second subject and transmits the calculated prediction to the prediction engine 212. The remote device 430 may then output the calculated prediction received from the venous thromboembolism prediction system 200.

In embodiments, the venous thromboembolism prediction system 200 can update the training data 202 based on the second values received for the second subjects, as well as the predicted venous thromboembolism outcomes. As such, the venous thromboembolism prediction system 200 can continually learn from new data regarding subjects. The venous thromboembolism prediction system 200 can store the predicted venous thromboembolism outcome with an association to the second value(s) received for the second subject in the training data 202. The predicted venous thromboembolism outcome may be stored with an indication of being a predicted value (as compared to the known venous thromboembolism outcomes for the plurality of first subjects), which can enable the machine learning engine 204 to process predicted outcome data stored in the training data 202 differently than known outcome data. In addition, it will be appreciated that over time, the second subject based on which a predicted outcome was generate may also have a known venous thromboembolism outcome (e.g., based on the onset of symptoms indicating that the second subject has venous thromboembolism, or based on an indication that the second subject does not have venous thromboembolism, such as a sufficient period of time passing subsequent to the generation of the predicted venous thromboembolism outcome). The venous thromboembolism venous thromboembolism system 200 can store the known venous thromboembolism outcome with an association to the second value(s) received for the second subject. The venous thromboembolism prediction system 200 can also store the known venous thromboembolism outcome with an indication of an update relative to the predicted venous thromboembolism outcome, which can enable the machine learning engine 204 to learn from the update and thus improve the feature selection and prediction processes used to generate and select the candidate machine learning model 114/subset of model parameters for use by the prediction engine 210. In embodiments, the venous thromboembolism prediction system 200 calculates a difference between the predicted venous thromboembolism outcome and the known venous thromboembolism outcome and stores this difference as the indication of the update.

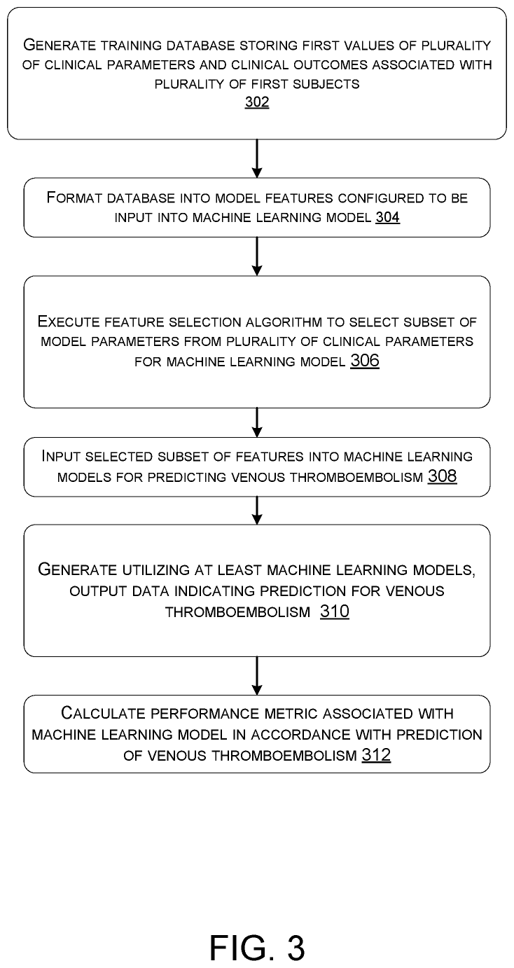

Referring now to FIG. 3, a process for predicting venous thromboembolism and flow of data that occurs in the machine learning engine 204. The process can be performed by various systems described herein, including the venous thromboembolism prediction system 200 and or the remote device 430. The training data 302 comprises biological data 102, injury severity data 104, subject data, and administration of blood products data 108.

In embodiments, preprocessing is executed on the training data. Pre-processing may be performed before feature selection 306 and/or the data are fed into machine learning models. In embodiments, an imputation algorithm can be executed to generate values for missing data in the training data 100. In embodiments, at least one of up-sampling or predictor rank transformations is executed on the data of the training database. Up-sampling and/or predictor rank transformation can be executed only for feature selection to accommodate class imbalance and non-normality in the data. While up-sampling or predictor rank transformations are discussed, many others are contemplated.

At feature selection 306, one or more feature selection algorithms in the feature selection component 112 are executed using the data stored in the training data to select, for each machine learning model. The subsets of model parameters are selected from the plurality of clinical parameters of the training data 100, such that a count of each subset of model parameters is less than a count of the clinical parameters. Feature selection machine learning engines such as backwards variable elimination constraint-based algorithms, constrain-based structure learning algorithms, and/or constraint-based local discovery learning algorithms can be used to select the subsets of model parameters. For example, the machine learning engine 204 can execute machine learning algorithms from the “bnlearn” R package, including but not limited to the Grow-Shrink (“gs”), Incremental Association Markov Blanket (“iamb”), Fast Incremental Association (“fast.iamb”), Max-Min Parents & Children (“mmpc”), or Semi-Interleaved Hiton-PC (“si.hiton.pc”) algorithms. While these feature selection algorithms are enumerated, many others are contemplated. In embodiments, the clinical parameters are randomly re-ordered prior to feature selection.

At data formatting 304, the training data 100 is formatted to be fed into the machine learning algorithm. Data formatting may include standardization of data, scaling of data, imputing data using missing data algorithms, or other forms of data formatting. At inputting the selected subset of features into machine learning models for predicting venous thromboembolism 308, the data is fed into the machine learning models 114 either through cloud-based approaches or via local networks.

At generate utilizing at least machine learning models, output data indicating prediction for venous thromboembolism 310 the machine learning models may be executed by identifying first values of clinical parameters in the training database corresponding to each subset of model parameters, and generating predictions of venous thromboembolism outcomes using the identified first values. In embodiments, the prediction algorithms include a plurality of linear discriminant analysis (lda), classification and regression trees (cart), k-nearest neighbors (knn), support vector machine (svm), logistic regression (glm), random forest (rt), generalized linear models (glmnet) and/or naïve Bayes (nb) algorithms. While these algorithms for the classification machine learning models are discussed, many more are contemplated.

At calculate performance metric associated with machine learning model in accordance with prediction of venous thromboembolism, 312 at least one performance metric is calculated for each prediction machine learning model (e.g., each combination of (i) a subset of model parameters selected using a feature selection machine learning model and (ii) a prediction machine learning model used to generate one or more clinical outcome predictions). The performance metrics can represent the ability of each combination to predict venous thromboembolism. The performance metric can include at least one of an AUC score, a sensitivity, or a specificity. In embodiments, a ROC curve can be generated based on the sensitivity and the specificity. An AUC can be calculated based on the ROC curve. In embodiments, the candidate prediction machine learning model can be evaluated by further performance metrics. For example, the candidate prediction machine learning model can be evaluated based on Accuracy, No Information Rate, positive predictive value and negative predictive value. While these performance metrics are discussed, many more are contemplated. Then, a candidate prediction machine learning algorithm is selected based on the performance metric(s). Various policies, heuristics, or other rules can be applied based on the performance metric(s) to select a candidate machine learning model (and corresponding subset of model parameters selected by one of the feature selection algorithms). For example, values for each performance metrics can be compared to respective threshold values, and a machine learning model can be determined to be a candidate machine learning model (or a potential candidate) responsive to the value for the performance metric exceeding the threshold. In embodiments, the candidate prediction machine learning model and corresponding subset of model parameters are selected based on the rule: identify the combination having (1) a highest AUC score; subsequently, (2) a highest sensitivity; and (3) subsequently, a specificity greater than a threshold specificity.

In embodiments, the methods described herein involve five major components: (1) initial data exploration, (2) variable and/or feature selection, (3) prediction, (4) model selection, (5) deployment and self-improvement. It will be understood by those possessing ordinary skill in the art that these stages may not be discrete entities and there may be overlap between them, and that the output from each stage may be used to inform, calibrate, and/or improve other stages of the engine and the machine learning engine. To perform feature selection on an entire set of clinical parameters, backwards variable elimination may be used. Feature selection may be performed by removing variables that are highly correlated. In embodiments where subjects have an injury (such as an injury that puts them at risk for venous thromboembolism), summations of wound volume and wound surface area can be added to the variable set to account for patient wound burden. One or more of up-sampling, data imputation, and predictor rank transformations can be performed to improve feature selection and accommodate class imbalance in the data. The variable sets can be run in sundry binary prediction algorithms, and the best variable set and binary prediction algorithm combination that firstly produces the highest AUC score and then the highest Sensitivity and reasonable Specificity can be chosen. Optionally, the resultant models can be examined using Accuracy, No Information Rate, positive predictive value and negative predictive value. Optionally, model performance can be further assessed Decision Curve Analysis (DCA).

In embodiments, comparisons of the feature selected models to the full variable models shows better performance in the former. This is a strength of the methods described herein, since over-parameterization frequently leads to model underperformance. In variable selected models as described herein, the ROC curves and their respective AUCs show that the models have good predictive ability. Similarly, these models have higher Accuracy and Kappa statistics than the full variable models.

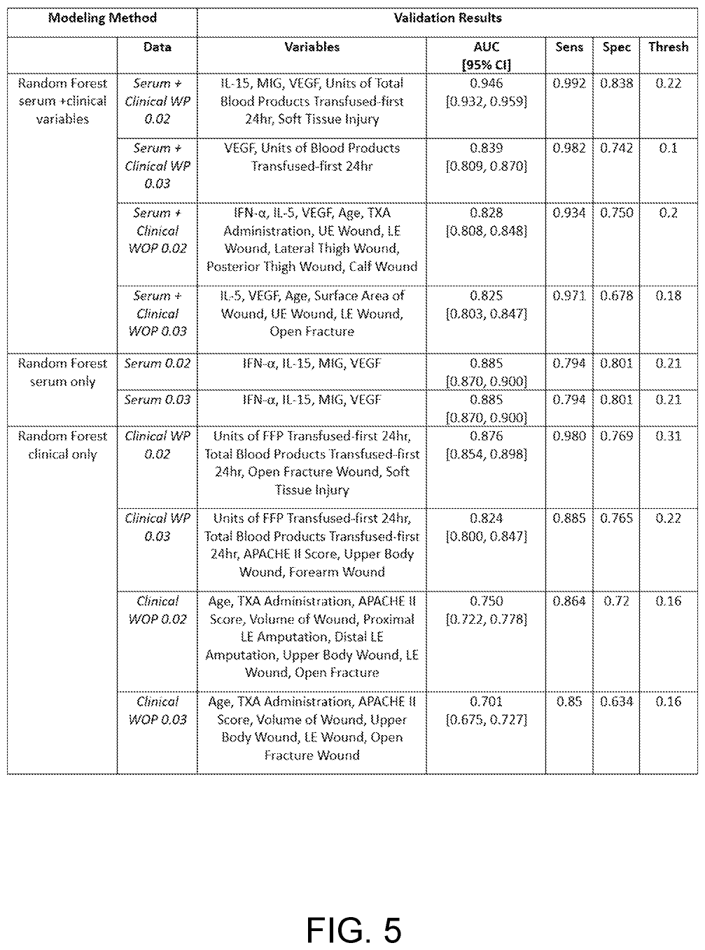

FIG. 5 illustrates an example of clinical parameters with differential values that drive the feature selection, a feature selection result that depicts the importance of each variable on the fit and prediction capacity of the model, and a performance metric that evaluates the accuracy, sensitivity, and specificity of prediction. Clinical parameters with differential values from an analysis of clinical parameters obtained in a study of wound closure are shown.

In embodiments, the methods disclosed herein relate to determining a subject's risk profile for venous thromboembolism, determining if a subject has an increased risk of developing venous thromboembolism, assessing risk factors in a subject, detecting levels of biomarkers, and treating a subject for venous thromboembolism. In accordance with any embodiments of the methods described herein, the subject may be assessed prior to the detection of symptoms of venous thromboembolism, such as prior to detection of symptoms of venous thromboembolism. In accordance with any embodiments of the methods described herein, the test subject may be assessed prior to the onset of any detectable symptoms of venous thromboembolism, such as prior to the subject having symptoms of venous thromboembolism detectable by one or more such methodologies. In accordance with any embodiments of the methods described herein, the test subject may have an injury, condition, or wound that puts the subject at risk of developing venous thromboembolism, such as a blast injury, a crush injury, a gunshot wound, or an extremity wound.

In embodiments, there are provided methods of assessing risk factors (e.g., clinical parameters) in a subject, the methods comprising, consisting of, or consisting essentially of measuring, assessing, detecting, assaying, and/or determining one or more clinical parameters, such as one or more selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-1α) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNFα) in a sample from the subject, level of vascular endothelial growth factor (VEGF) in a sample from the subject, amount of whole blood cells administered to the subject, amount of red blood cells (RBCs) administered to the subject, amount of packed red blood cells (PRBCs) administered to the subject, amount of platelets administered to the subject, units of blood products transfused in the first 24 hours, summation of all blood products administered to the subject, level of total packed RBCs, Injury Severity Score (ISS), Abbreviated injury scale (AIS) of abdomen, AIS of chest (thorax), AIS of extremity, AIS of face, AIS of head, and AIS of skin. In particular embodiments, there are provided methods of assessing risk factors (e.g., clinical parameters) in a subject, the methods comprising, consisting of, or consisting essentially of measuring, assessing, detecting, assaying, and/or determining one or more clinical parameters, such as one or more selected from AIS of head in the subject, AIS of abdomen in the subject, amount of platelets administered to the subject, level of total packed RBCs administered to the subject, summation of all blood products administered to the subject, level of IP-10 in a serum sample from the subject, level of IL-10 in a serum sample from the subject, and level of MCP-1 in a serum sample from the subject.

In embodiments, there are provided methods of detecting levels of biomarkers, the methods comprising, consisting of, or consisting essentially of measuring, detecting, assaying, or determining in one or more samples from the subject levels of one or more biomarkers selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-1α) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNF-α) in a sample from the subject, level of vascular endothelial growth factor (VEGF) in a sample from the subject.

In embodiments, there are provided methods of detecting levels of biomarkers, the methods comprising, consisting of, or consisting essentially of measuring, detecting, assaying, or determining in one or more samples from the subject levels of one or more biomarkers selected from IL-15, VEGF and MIG. In embodiments, the one or more biomarkers comprise, consist of, or consist essentially of levels of IL-15, VEGF and MIG.

In embodiments, there are provided methods for predicting venous thromboembolism in a subject comprising assaying levels of biomarkers. In embodiments, the method comprises obtaining a biological sample from the subject, assaying biomarkers in the biological sample and predicting venous thromboembolism in the subject. In embodiments, the biomarkers include IL-15, MIG, and VEGF and the method of assaying includes measuring or detecting biomarkers such as IL-15, MIG, and VEGF. Moreover, the method includes measuring or detecting total number of blood products transfused in the first 24 hours in addition to measuring or detecting biomarkers. Further, the method includes assessing soft tissue injury in addition to measuring or detecting biomarkers and/or total number of blood products transfused in first 24 hours. Furthermore, the method includes treating the subject for venous thromboembolism, as described herein, in addition to measuring or detecting biomarkers, measuring or detecting total number of blood products, and/or assessing soft tissue injury.

In embodiments, one or more clinical parameters, two or more clinical parameters, three or more clinical parameters, four or more clinical parameters, five or more clinical parameters, six or more clinical parameters, seven or more clinical parameters, eight or more clinical parameters, nine or more clinical parameters, ten or more clinical parameters, 11 or more clinical parameters, 12 or more clinical parameters, 13 or more clinical parameters, 14 or more clinical parameters, 15 or more clinical parameters, 16 or more clinical parameters, 17 or more clinical parameters, 18 or more clinical parameters, 19 or more clinical parameters, 20 or more clinical parameters, 21 or more clinical parameters, 22 or more clinical parameters, 23 or more clinical parameters, 24 or more clinical parameters, 25 or more clinical parameters, 26 or more clinical parameters, 27 or more clinical parameters, 28 or more clinical parameters, 29 or more clinical parameters, 30 or more clinical parameters, 31 or more clinical parameters, 32 or more clinical parameters, 33 or more clinical parameters, 34 or more clinical parameters, 35 or more clinical parameters, 36 or more clinical parameters, 37 or more clinical parameters, 38 or more clinical parameters, 39 or more clinical parameters, 40 or more clinical parameters, 41 or more clinical parameters, 42 or more clinical parameters, 43 or more clinical parameters, 44 or more clinical parameters, 45 or more clinical parameters, such as selected from those set forth above are measured, assessed, detected, assayed, and/or determined. In particular embodiments, 2, 3, 4, 5, 6, 7, or 8 clinical parameters are measured, assessed, detected, assayed, and/or determined.

To assay, detect, measure, and/or determine levels of individual clinical parameters, one or more samples is taken or isolated from the subject. In embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 samples are taken or isolated from the subject. The one or more samples may or may not be processed prior assaying levels of the factors, risk factors, biomarkers, clinical parameters, and/or components. For example, whole blood may be taken from an individual and the blood sample may be processed, e.g., centrifuged, to isolate plasma or serum from the blood. The one or more samples may or may not be stored, e.g., frozen, prior to processing or analysis. In embodiments, one or more clinical parameters selected from are detected in a sample from a subject that is not a serum sample, such as wound effluent.

In embodiments, levels of individual biomarkers in a sample isolated from a subject are assessed, detected, measured, and/or determined using mass spectrometry in conjunction with ultra-performance liquid chromatography (UPLC), high-performance liquid chromatography (HPLC), gas chromatography (GC), gas chromatography/mass spectroscopy (GC/MS), or UPLC. Other methods of assessing biomarkers include biological methods, such as but not limited to ELISA assays, Western Blot, and multiplexed immunoassays. Other techniques may include using quantitative arrays, PCR, RNA sequencing, DNA sequencing, and Northern Blot analysis. Other techniques include Luminex proteomic data, RNAseq, transcriptomic data, quantitative polymerase chain reaction (qPCR) data, microarray, and quantitative bacteriology data.

In embodiments, the biomarkers include proteins and nucleic acids isolated from biological samples, for example tissue, organ, or biological fluids of a subject. Examples of biological fluids include blood, serum, plasma, sweat, urine, saliva, peritoneal fluid, wound effluent, and spinal fluid.

The present disclosure also describes microarrays including biomarkers comprising proteins or nucleic acids for predicting venous thromboembolism. In embodiments, proteins and nucleic acids can be linked to chips, such as microarray chips (see U.S. Pat. Nos. 6,040,138 and 7,148,058). Binding to proteins or nucleic acids on microarrays can be detected by scanning the microarray with a variety of laser or charge coupled device (CCD)-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.32.), or GenePix (Axon Instruments). A microarray panel including one or more biomarkers for a clinical outcome can be used for predicting the risk of a subject in developing a clinical outcome and/or for monitoring subject undergoing treatment for a clinical outcome.

To determine levels of clinical parameters, particularly biomarkers, it is not necessary that an entire biomarker molecule, e.g., a full-length protein or an entire RNA transcript, be present or fully sequenced. In other words, determining levels of, for example, a fragment of protein being analyzed may be sufficient to conclude or assess that an individual component of the risk profile being analyzed is increased or decreased. Similarly, if, for example, arrays or blots are used to determine biomarker levels, the presence, absence, and/or strength of a detectable signal may be sufficient to assess levels of biomarkers.

Biomarkers can be detected, assayed, or measured using the Luminex™ immune assay platform, available from ThermoFisher Scientific. For example the Cytokine & Chemokine 34-Plex Human ProcartaPlex™ Panel 1A (cat #EPX340-12167-901) detects the following targets in a single serum or plasma sample: Eotaxin/CCL11; GM-CSF; GRO alpha/CXCL1; IFN alpha; IFN gamma; IL-1 beta; IL-1 alpha; IL-1RA; IL-2; IL-4; IL-5; IL-6; IL-7; IL-8/CXCL8; IL-9; IL-10; IL-12 p70; IL-13; IL-15; IL-17A; IL-18; IL-21; IL-22; IL-23; IL-27; IL-31; IP-10/CXCL10; MCP-1/CCL2; MIP-1 alpha/CCL3; MIP-1 beta/CCL4; RANTES/CCL5; SDF1 alpha/CXCL12; TNF alpha; TNF beta/LTA. While this set of markers and this platform is discussed, it is known to persons having ordinary skill in the art that additional markers may be measured using similar types of platforms (e.g., other multiplex assays).

In embodiments, kits are used to measure at least one of the level of clinical parameters and biomarkers. The kits can include microarrays, ELISA assays, multiplex assays, reagents for sample preparation and assay completion. The kits can also include one or more computer systems, hardware for detection, and other components. In one aspect, the invention features a kit that includes a device having an immobilized binding agent on its surface in a pattern that generates a signal, wherein the immobilized binding agent is capable of binding to a biomarker complex. The kit also includes a detecting binding agent that specifically binds to a first component of the biomarker complex, a detecting binding agent that specifically binds to a second component of the biomarker complex, and a detecting binding agent that specifically binds to a third component of the biomarker complex. The detecting binding agents that specifically bind to the first, second, or third component of the biomarker complex do not specifically bind to any other component of the biomarker complex.

In another aspect, the invention features a kit that includes a device having an immobilized binding agent on its surface in a pattern that generates a signal, wherein the immobilized binding agent is capable of binding to a biomarker. The kit also includes at least three detecting binding agents that specifically bind to different epitopes of the biomarker. An example of a biomarker or a component of a biomarker complex is a protein, a nucleic acid, a virus, or a cell. The amount, e.g., presence, absence, or concentration (relative or absolute), of a component of the biomarker complex present in a biological sample may be an indicator of a disease (e.g., venous thromboembolism) in a subject, or the ratio (e.g., relative amount) between the components may be an indicator of a disease (e.g., venous thromboembolism) in a subject. The presence, absence, or degree of posttranslational modification, alternative splicing, or degradation of a biomarker present in the biological sample may also be an indicator of a disease in a subject. The biomarker complex may be an immune complex or biomarker complex, or multiple biomarkers and biomarker complexes may be detected in a biological sample using the methods and kits described herein.

In embodiments, clinical parameters are detected, measured, assayed, assessed, and/or determined in a sample isolated from the subject at different time points, such as before, at a first time point after, and/or at a subsequent time point after the subject contracts an injury, condition, or wound that puts the subject at risk of developing venous thromboembolism, such as a blast injury, a crush injury, a gunshot wound, or an extremity wound. For example, embodiments of the methods described herein may comprise detecting biomarkers at two, three, four, five, six, seven, eight, nine, 10 or even more time points over a period of time, such as a week or more, two weeks or more, three weeks or more, four weeks or more, a month or more, two months or more, three months or more, four months or more, five months or more, six months or more, seven months or more, eight months or more, nine months or more, ten months or more, 11 months or more, a year or more or even two years or longer. The methods also include embodiments in which the subject is assessed before and/or during and/or after treatment for venous thromboembolism. In embodiments, the methods are useful for monitoring the efficacy of treatment of venous thromboembolism, and comprise detecting clinical parameters, such as biomarkers in a sample isolated from the subject, at least one, two, three, four, five, six, seven, eight, nine or 10 or more different time points prior to beginning treatment for venous thromboembolism and subsequently detecting clinical parameters, such as at least one, two, three, four, five, six, seven, eight, nine or 10 or more different time points after beginning of treatment for venous thromboembolism, and determining the changes, if any, in the levels detected. The treatment may be any treatment designed to cure, remove or diminish the symptoms and/or cause(s) of venous thromboembolism.

In embodiments, there are provided methods of detecting clinical parameters in a subject, the method comprising, consisting of, or consisting essentially of measuring levels of one or more clinical parameters selected from soft tissue injury, units total blood products transfused in first 24 hours, and serum levels of vascular endothelial growth factor (VEGF), monokine induced by gamma interferon (MIG), and interleukin 15 (IL-15). In embodiments, the methods comprise detecting elevated levels. As used herein, “elevated” refers to a level or value that is increased relative to a reference level or value. As used herein, “reduced” refers to a level or value that is reduced relative to a reference level or value. In embodiments, the reference value is a value previously detected, measured, assayed, assessed, or determined for the subject. In embodiments, the reference value is detected, measured, assayed, assessed, or determined for a population of one or more reference subjects at a time when the reference subjects did not have detectable symptoms of venous thromboembolism.

In embodiments, there are provided methods of determining a risk profile for venous thromboembolism, wherein the risk profile comprises, consists of, or consists essentially of one or more components based on one or more clinical parameters selected from level of epidermal growth factor (EGF) in a sample from the subject, level of eotaxin-1 (CCL11) in a sample from the subject, level of basic fibroblast growth factor (bFGF) in a sample from the subject, level of granulocyte colony-stimulating factor (G-CSF) in a sample from the subject, level of granulocyte-macrophage colony-stimulating factor (GM-CSF) in a sample from the subject, level of hepatocyte growth factor (HGF) in a sample from the subject, level of interferon alpha (IFN-α) in a sample from the subject, level of interferon gamma (IFN-γ) in a sample from the subject, level of interleukin 10 (IL-10) in a sample from the subject, level of interleukin 12 (IL-12) in a sample from the subject, level of interleukin 13 (IL-13) in a sample from the subject, level of interleukin 15 (IL-15) in a sample from the subject, level of interleukin 17 (IL-17) in a sample from the subject, level of interleukin 1 alpha (IL-1α) in a sample from the subject, level of interleukin 1 beta (IL-1β) in a sample from the subject, level of interleukin 1 receptor antagonist (IL-1RA) in a sample from the subject, level of interleukin 2 (IL-2) in a sample from the subject, level of interleukin 2 receptor (IL-2R) in a sample from the subject, level of interleukin 3 (IL-3) in a sample from the subject, level of interleukin 4 (IL-4) in a sample from the subject, level of interleukin 5 (IL-5) in a sample from the subject, level of interleukin 6 (IL-6) in a sample from the subject, level of interleukin 7 (IL-7) in a sample from the subject, level of interleukin 8 (IL-8) in a sample from the subject, level of interferon gamma induced protein 10 (IP-10) in a sample from the subject, level of monocyte chemoattractant protein 1 (MCP-1) in a sample from the subject, level of monokine induced by gamma interferon (MIG) in a sample from the subject, level of macrophage inflammatory protein 1 alpha (MIP-1α) in a sample from the subject, level of macrophage inflammatory protein 1 alpha (MIP-1β) in a sample from the subject, level of chemokine (C-C motif) ligand 5 (CCL5) in a sample from the subject, level of tumor necrosis factor alpha (TNF-α) in a sample from the subject, level of vascular endothelial growth factor (VEGF) in a sample from the subject, amount of whole blood cells administered to the subject, amount of red blood cells (RBCs) administered to the subject, amount of packed red blood cells (PRBCs) administered to the subject, amount of platelets administered to the subject, summation of all blood products administered to the subject, units of total blood products transfused in first 24 hours, level of total packed RBCs, Injury Severity Score (ISS), Abbreviated injury scale (AIS) of abdomen, AIS of chest (thorax), AIS of extremity, AIS of face, AIS of head, AIS of skin, and soft tissue injury. Such methods may comprise, consist of or consist essentially of detecting the one or more clinical parameters for the subject, and calculating the subject's risk profile value from the detected clinical parameters.

In embodiments, the risk profile is calculated from one or more clinical parameters, two or more clinical parameters, three or more clinical parameters, four or more clinical parameters, five or more clinical parameters, six or more clinical parameters, seven or more clinical parameters, eight or more clinical parameters, nine or more clinical parameters, ten or more clinical parameters, 11 or more clinical parameters, 12 or more clinical parameters, 13 or more clinical parameters, 14 or more clinical parameters, 15 or more clinical parameters, 16 or more clinical parameters, 17 or more clinical parameters, 18 or more clinical parameters, 19 or more clinical parameters, 20 or more clinical parameters, 21 or more clinical parameters, 22 or more clinical parameters, 23 or more clinical parameters, 24 or more clinical parameters, 25 or more clinical parameters, 26 or more clinical parameters, 27 or more clinical parameters, 28 or more clinical parameters, 29 or more clinical parameters, 30 or more clinical parameters, 31 or more clinical parameters, 32 or more clinical parameters, 33 or more clinical parameters, 34 or more clinical parameters, 35 or more clinical parameters, 36 or more clinical parameters, 37 or more clinical parameters, 38 or more clinical parameters, 39 or more clinical parameters, 40 or more clinical parameters, 41 or more clinical parameters, 42 or more clinical parameters, 43 or more clinical parameters, 44 or more clinical parameters, 45 or more clinical parameters, such as selected from those set forth above. In particular embodiments, the risk profile is calculated from 2, 3, 4, 5, 6, 7, or 8 clinical parameters such as selected from those set forth above. In embodiments, a subject is diagnosed as having an increased risk of suffering from venous thromboembolism if the subject's five, four, three, two or even one of the components or factors herein are at abnormal levels. It should be understood that individual levels of risk factor need not be correlated with increased risk in order for the risk profile value to indicate that the subject has an increased risk of developing venous thromboembolism. In embodiments, one or more clinical parameters selected from the panel of biomarkers listed above are detected in a sample from a subject that is not a serum sample, such as wound effluent, or other biological fluids.

In embodiments, one or more clinical parameters are detected in a sample from the subject that is a biological fluid or tissue isolated from the subject. Biological fluids or tissues include but are not limited to whole blood, peripheral blood, serum, plasma, cerebrospinal fluid, wound effluent, urine, amniotic fluid, peritoneal fluid, lymph fluids, various external secretions of the respiratory, intestinal, and genitourinary tracts, tears, saliva, white blood cells, solid tumors, lymphomas, leukemias, and myelomas. In embodiments, one or more clinical parameters are detected in a sample from the subject selected from a serum sample and wound effluent. In embodiments, the sample is a plasma sample from the subject.

In embodiments, the measurements of the individual components themselves are used in the risk profile, and these levels can be used to provide a “binary” value to each component, e.g., “elevated” or “not elevated.” Each of the binary values can be converted to a number, e.g., “1” or “0,” respectively.

In embodiments, the “risk profile value” can be a single value, number, factor or score given as an overall collective value to the individual components of the profile. For example, if each component is assigned a value, such as above, the component value may simply be the overall score of each individual or categorical value. For example, if four components of the risk profile for predicting venous thromboembolism are used and three of those components are assigned values of “+2” and one is assigned values of “+1,” the risk profile in this example would be +7, with a normal value being, for example, “0.” In this manner, the risk profile value could be a useful single number or score, the actual value or magnitude of which could be an indication of the actual risk of developing venous thromboembolism, e.g., the “more positive” the value, the greater the risk of developing venous thromboembolism.

In embodiments, the “risk profile value” can be a series of values, numbers, factors or scores given to the individual components of the overall profile. In embodiments, the “risk profile value” may be a combination of values, numbers, factors or scores given to individual components of the profile as well as values, numbers, factors or scores collectively given to a group of components, such as a plasma marker portion. In another example, the risk profile value may comprise or consist of individual values, number or scores for specific component as well as values, numbers or scores for a group of components.

In embodiments, individual values from the risk profile can be used to develop a single score, such as a “combined risk index,” which may utilize weighted scores from the individual component values reduced to a diagnostic number value. The combined risk index may also be generated using non-weighted scores from the individual component values. In such embodiments, when the “combined risk index” exceeds a specific threshold level, such as may be determined by a range of values developed similarly from a population of one or more control (normal) subjects, the individual may be deemed to have a high risk, or higher than normal risk, of developing venous thromboembolism, whereas maintaining a normal range value of the “combined risk index” would indicate a low or minimal risk of developing venous thromboembolism. In these embodiments, the threshold value may be set by the combined risk index from a population of one or more control (normal) subjects.

In embodiments, the value of the risk profile can be the collection of data from the individual measurements, and need not be converted to a scoring system, such that the “risk profile value” is a collection of the individual measurements of the individual components of the profile.

In embodiments, the subject's risk profile is compared to a reference risk profile. In embodiments, the reference risk profile value is calculated from clinical parameters previously detected for the subject. Thus, the present application also includes methods of monitoring the progression of venous thromboembolism in a subject, with the methods comprising determining the subject's risk profile at more than one time point. For example, embodiments of the methods of the present application will comprise determining the subject's risk profile at two, three, four, five, six, seven, eight, nine, 10 or even more time points over a period of time, such as a week or more, two weeks or more, three weeks or more, four weeks or more, a month or more, two months or more, three months or more, four months or more, five months or more, six months or more, seven months or more, eight months or more, nine months or more, ten months or more, 11 months or more, a year or more or even two years or longer. The methods described herein also include embodiments in which the subject's risk profile is assessed before and/or during and/or after treatment of venous thromboembolism. In other words, the present application also includes methods of monitoring the efficacy of treatment of venous thromboembolism by assessing the subject's risk profile over the course of the treatment and after the treatment. In embodiments, the methods of monitoring the efficacy of treatment of venous thromboembolism comprise determining the subject's risk profile at least one, two, three, four, five, six, seven, eight, nine or 10 or more different time points prior to the receipt of treatment for venous thromboembolism and subsequently determining the subject's risk profile at least one, two, three, four, five, six, seven, eight, nine or 10 or more different time points after beginning of treatment for venous thromboembolism, and determining the changes, if any, in the risk profile of the subject. The treatment may be any treatment designed to cure, remove or diminish the symptoms and/or cause(s) of venous thromboembolism.

In embodiments, the reference risk profile value is calculated from clinical parameters detected for a population of one or more reference subjects when the reference subjects did not have detectable symptoms of venous thromboembolism. In embodiments, the reference risk profile value is calculated from clinical parameters detected for a population of reference subjects having an injury, condition, or wound that puts the subject at risk of developing venous thromboembolism, such as a blast injury, a crush injury, a gunshot wound, or an extremity wound.

The levels or values of the clinical parameters compared to reference levels can vary. In embodiments, the levels or values of any one or more of the factors, risk factors, biomarkers, clinical parameters, and/or components is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000 fold higher than reference levels or values. In embodiments, the levels or values of any one or more of the factors, risk factors, biomarkers, clinical parameters, and/or components is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 30, 40, 50, 60, 70, 80, 90, 100, 500, 1,000, or 10,000 fold lower than reference levels or values. In the alternative, the levels or values of the factors or components may be normalized to a standard and these normalized levels or values can then be compared to one another to determine if a factor or component is lower, higher or about the same.

In embodiments, an increase in the subject's risk profile value as compared to a reference risk profile value indicates that the subject has an increased risk of developing venous thromboembolism.

In embodiments, the subject's risk profile is compared to the profile that is deemed to be a “normal” risk profile. To establish a “normal” risk profile, an individual or group of individuals may be first assessed to ensure they have no signs, symptoms or diagnostic indicators that they may have venous thromboembolism. Then, the risk profile of the individual or group of individuals can then be determined to establish a “normal risk profile.” In one embodiment, a normal risk profile can be ascertained from the same subject when the subject is deemed healthy, such as when the subject does not have an injury, condition, or wound that puts the subject at risk of developing venous thromboembolism, such as a blast injury, a crush injury, a gunshot wound, or an extremity wound and/or has no signs, symptoms or diagnostic indicators of venous thromboembolism. In embodiments, however, a risk profile from a “normal subject,” e.g., a “normal risk profile,” is from a subject who has an injury or wound but has no signs, symptoms or diagnostic indicators that they may have venous thromboembolism, such as a subject who has a chest wound, but has no signs, symptoms or diagnostic indicators of venous thromboembolism, or a head wound but no signs, symptoms or diagnostic indicators of venous thromboembolism, or has at least one wound in an extremity (arm, hand, finger(s), leg, foot, toe(s)), but no signs, symptoms or diagnostic indicators of venous thromboembolism. Thus, in embodiments, a “normal” risk profile is assessed in the same subject from whom the sample is taken, prior to the onset of any signs, symptoms or diagnostic indicators that they have venous thromboembolism. For example, the normal risk profile may be assessed in a longitudinal manner based on data regarding the subject at an earlier point in time, enabling a comparison between the risk profile (and values thereof) over time.

In embodiments, a normal risk profile is assessed in a sample from a different subject or patient (from the subject being analyzed) and this different subject does not have or is not suspected of having venous thromboembolism. In embodiments, the normal risk profile is assessed in a population of healthy individuals, the constituents of which display no signs, symptoms or diagnostic indicators that they may have venous thromboembolism. Thus, the subject's risk profile can be compared to a normal risk profile generated from a single normal sample or a risk profile generated from more than one normal sample.

In embodiments, such as for univariate analysis, a Wilcoxon rank-sum test can be used to identify which biomarkers from specific patient groups are associated with a specific indication. The assessment of the levels of the individual components of the risk profile can be expressed as absolute or relative values and may or may not be expressed in relation to another component, a standard, an internal standard or another molecule or compound known to be in the sample. If the levels are assessed as relative to a standard or internal standard, the standard or internal standard may be added to the test sample prior to, during or after sample processing.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “engine,” “module,” or “system.” Furthermore, aspects of the present disclosure may take the form of a computer program product embodied in one or more computer readable medium(s) having computer readable program code embodied thereon. Aspects of the present disclosure may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various input/output, control, analysis and other functions described herein, such as by executing instructions of a computer program product.

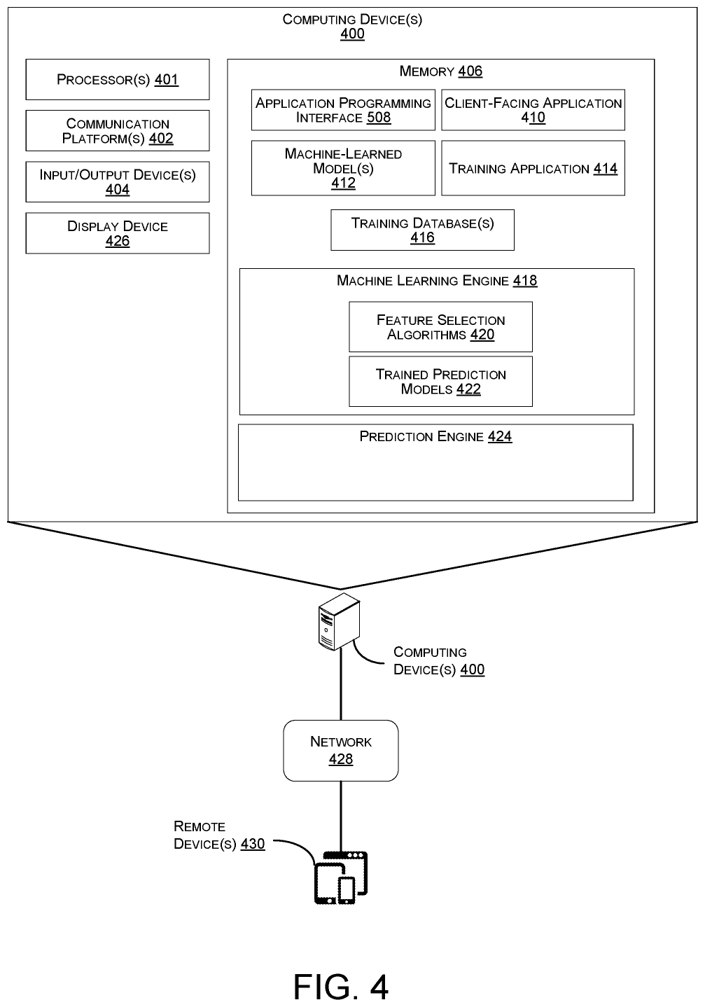

In embodiments, the computer device, computer readable media, network, and remote device may be arranged in the architecture depicted in FIG. 4. The computing device 400 houses at least, but is not limited to a processor(s) 401, communication platform(s) 402, input/output device(s) 404, memory 406, a machine learning engine 418, and a prediction engine 424. The memory includes at least, but is not limited to an application programming interface 408, a client-facing application 410, machine learned models 412, training application 414, and a training database 416, a machine learning engine 518 that comprises feature selection algorithms 420, trained prediction models 422, and a display device 426. The memory also includes a prediction engine 424.

In embodiments, the computer devices are tethered to a remote device 430 through a network 428. The network enables communication via internet with a secure and protected host website operating the model algorithm and providing an output after predictive variables are entered.

In embodiments, the communication platform 402 can include communication electronics, wherein the communications electronics can be configured to transmit and receive electronic signals from a remote source, such as another electronic device, a cloud server, or an Internet resource. The communications electronics can be configured to communicate using any number or combination of communication standards (e.g., Bluetooth, GSM, CDMA, TDNM, WCDMA, OFDM, GPRS, EV-DO, Wi-Fi, WiMAX, S02.xx, UWB, LTE, satellite). The communications electronics may also include wired communications features, such as USB ports, serial ports, IEEE 1394 ports, optical ports, parallel ports, and/or any other suitable wired communication port.

In embodiments, the input/output device(s) 404 may include one or more of a computer, a keyboard, a mouse, a mobile device (e.g., a mobile phone, a tablet, a laptop), a screen, a microphone, or a printing device. The user input device can include various user interface elements such as keys, buttons, sliders, knobs, touchpads (e.g., resistive or capacitive touchpads), or microphones. In embodiments, the user interface device includes a touchscreen display device and user input device, such that the user interface device can receive user inputs as touch inputs and determine commands indicated by the user inputs based on detecting location, intensity, duration, or other parameters of the touch inputs.

In embodiments, the application programming interface 408 and the client-facing application 410 may be implemented using various software environments, including but not limited to SAS and R package. SAS (“statistical analysis software”) is a general-purpose package (similar to Stata and SPSS) created by Jim Goodnight and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis. R package is free, general purpose package that complies with and runs on a variety of UNIX platforms.

Any combination of one or more computer readable medium(s) may be utilized to store the machine-learned models 412, the training application 414, and the training database 416. The one or more computer readable medium(s) may also be utilized to store the machine learning engine 418 and the feature selection algorithms 420, the trained prediction models 422, the prediction machine learning model 424. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object-oriented programming language such as Java, Smalltalk, C++ or the like and conventional procedural programming languages, such as the “C” programming language or similar programming languages. The program code may execute entirely on the users computer, partly on the users computer, as a stand-alone software package, partly on the users computer and partly on a remote computer or entirely on the remote computer or server. In the latter scenario, the remote computer may be connected to the users computer through any type of network, including a local area network (LAN) or a wide area network (WAN), or the connection may be made to an external computer (for example, through the Internet using an Internet Service Provider).

Aspects of the present disclosure are described herein with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ± any percentage between 1% and 20% of the stated value.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXAMPLES

### Example 1

### Prediction of Venous Thromboembolism Using Clinical and Serum Biomarker Data from Trauma Patients

Venous thromboembolism (VTE) is a common occurrence in trauma patients with an estimated incidence of as high at 9% in the civilian literature and up to 28% in military combat casualties (Karcutskie et al., JAMA Surg. 152(1):35-40. 2017; Hannon et al., Am J Surg. 212(2):230-234, 2016) The consequences of VTE are not inconsequential as pulmonary embolism (PE) represents the third-leading cause of in-hospital deaths in trauma patients and is a leading cause of readmission (Rogers. Surgery. 130(1):1-12, 2001). In addition, VTE can lead to prolonged hospital stay and incur substantial economic burden with an estimated cost to the U.S. healthcare system of at least $7-12 billion each year (Paydar et al., Bull Emerg Trauma. 4(1):1-7, 2016). For the military healthcare system, VTE presents additional challenges with its high incidence and added risk factors such as above-knee amputations and prolonged immobilization from intercontinental aeromedical transport due (Grosse et al., Thomb Res. 137:3-10, 2016). Although VTE prophylaxis exists, identifying high-risk patients can be challenging because patients may be asymptomatic, may have a contraindication to chemical prophylaxis due to hemorrhage risk, or may be in multiorgan organ failure and too sick to undergo imaging studies such as a contrast-enhanced computed tomography (O'Donnell and Weitz, Can J Surg. 46(2):129-135, 2003). Additionally, even with the initiation of prophylaxis, it does not completely eliminate the development of VTE (Rogers et al., J Trauma. 53(1):142-164, 2002; Guyatt et al., Chest. 141(2 Suppl): 7S-47S, 2012). Thus, a predictive tool for assessing risk of VTE would have considerable value in potentially reducing patient morbidity and mortality while decreasing costs of VTE management.

Development of clinical decision support tools (CDSTs) has become increasingly more robust in the healthcare field in the last decade, given the rise of data-driven medicine (Koutkias et al., Yearb Med Inform. 27(1):122-128, 2018). In 2013, the Surgical Critical Care Initiative (SC2i), a multi-institutional collaborative of military and civilian institutions, was established to produce biomarker-driven CDSTs to guide individualized care for complex and critically injured patients. This cooperative effort has resulted in a CDST called WounDx™ capable of predicting wound coverage success in military trauma patients (Forsberg et al., EBioMedicine. 2(9):1235-1242, 2015; Chromy et al., J Trans Med. 11:281, 2013). A similar CDST for VTE prediction using clinical and biological analyte-based data may likewise offer substantial benefit to military trauma patients. Such a CDST could augment the care of critically ill and injured patients by predicting the likelihood of VTE, potentially allowing for earlier prophylactic or therapeutic measures to be adopted prior to onset of VTE.

The purpose of this study was to develop prognostic models using techniques of Random Forest and Logistic Regression to identify patients at higher risk for VTE in a cohort of combat trauma patients using: (1) clinical measures alone (2) serum cytokines alone (3) clinical measures and serum cytokines. Ultimately, these results would serve as a stepping stone to creating a CDST. These methods were chosen in order to compare the efficacy of different modeling techniques in producing VTE classification models. Random Forest is a decision tree modeling technique that works effectively with a combination of categorical and numeric features while Logistic Regression is a widely used regression technique for binary classification models. It was hypothesized that advanced modeling could accurately predict those injured service members at highest risk for VTE.

Methods. Data Collection. This study of a cohort of 73 combat casualties injured in Iraq or Afghanistan and evacuated to a single Continental U.S. military treatment facility between 2007 and 2012 was approved by the Walter Reed National Military Medical Center (WRNMMC) Institutional Review Board (IRB). All casualties had at least one extremity wound and were prospectively enrolled in an observational study after admittance to WRNMMC (Forsberg et al., EBioMedicine. 2(9):1235-1242, 2015). The data set was collected from a cohort of 73 military patients injured in Iraq or Afghanistan and evacuated to a single Continental U.S. military treatment facility between 2007 and 2012. Prior to patient enrollment, institutional approval from WRNMMC's IRB was obtained.

Wounds were treated according to the standard of care at WRNMMC, and clinical data was collected from injury to discharge. Serum was collected at every operation, which followed institutional protocols and took place at least every 48 hours in the operating room (Chromy et al., J Trans Med. 11:281, 2013). The serum sample data collected at the patient's first surgical debridement were used for modeling (i.e. the first surgical debridement upon admission to WRNMMC after arriving from an overseas facility), which occurred a median of 5 days after injury. Serum cytokine biomarker levels were assayed using a Luminex 100/200 IS xMAP Bead Array Platform (Luminex, Austin, Tex.) and a Human Cytokine 30-plex panel kit supplemented with a 2-plex panel (Invitrogen; Cat. No LHC6003 and LCP0002, Grand Island, N.Y).

Variable Selection. There were 73 patient records and 179 variables in the preliminary data set. Variables with patient-identification information, with the same value for all patients (i.e. all patients were male), missing more than 20% of entries, not available before diagnosis, or not generalizable to a broader, non-military population (i.e. variables indicating the military operation in which the patient participated) were eliminated from the data set. This resulted in a modeling data set of 73 records and 115 variables, including the outcome variable of VTE.

Proxy variables were identified (i.e. highly related variables, such as variables encoding Abbreviated Injury Scale (AIS) and Injury Severity Score (ISS)) and generated an additional data set without proxies, in which only one variable from each set of correlated variables was included in the data set. Univariate t-tests using VTE as the outcome variable were used to identify which variable(s) from each group of proxy variables had a statistically significant association with the outcome variable (p<0.05). These identified variables were then included in the modeling data set. Where multiple variables or no variables had a significant association with VTE, the variable that encompassed the most clinical information was chosen. For instance, both the AIS score for skin and a variable encoding whether the ISS was greater than 15 were significantly associated with VTE outcome according to a univariate t-test. Since ISS is calculated based on the AIS codes of various body regions, the variable encoding whether ISS was greater than 15 was selected for inclusion in the modeling set. This process resulted in a data set without proxies consisting of the 73 patient records and 92 variables.

Finally, data sets were split into subsets containing clinical-only and serum-only variables. Including the VTE outcome variable, there were 83 variables in the full clinical-only data set, 60 variables in the clinical-only data set without proxies, and 33 variables in the serum-only data set. FIG. 5 lists the variables included in each data set. Model performance was based on each individual subset of variables as well as a combined clinical and serum data group.

Random Forest. Random Forest (RF) models were built using each data set (i.e. the clinical and serum data set with proxies, the clinical and serum data set without proxies, the clinical-only data set with proxies, the clinical-only data set without proxies, and the serum-only data set). During Random Forest modeling, backwards elimination was used as a feature selection technique. The ranger package in R was used for modeling (Wright. Journal of Statistical Software. 77(1):1-17, 2017). Variables in the data set were iterated through and retained for the next iteration only if the sum of sensitivity, specificity, and area under the curve (AUC) was decreased by a threshold amount after the variable was dropped. Threshold amounts of 0.02 and 0.03 were used.

Sensitivity, specificity, and AUC were calculated using the number of iterations of leave-one-pair-out cross validation that was necessary for convergence, with sensitivity and specificity taken at the threshold where their product was maximum. Convergence was defined as when the sum of the sensitivity, specificity, and AUC varied by less than 0.01 for 4 sequential iteration sizes, starting with 100 iterations and with a step size of 50. Leave-one-pair-out cross validation, where a test case and a test control are left out before a model is trained on remaining data, was used to assess model performance at each step of backwards variable elimination. Training data was up sampled to account for the imbalance in the low number of cases to controls (i.e. cases were randomly sampled until the number of cases equaled the number of controls).

Recursive feature elimination (RFE) was used along with backwards elimination to refine model features and performance. RFE is a feature selection algorithm which iteratively removes features that are least informative for classification (Gholami et al. Proc IEEE Eng Med Biol Soc. 2012:5258-5261, 2012; Guyon et al., Machine Learning, 46(1-3): 389-422, 2002). RFE was implemented by performing Random Forest backwards elimination recursively until there were no more changes in the final features selected. In other words, after iterating through all the variables during backwards elimination, subset was identified to only include the selected features and then repeated backwards elimination on this reduced data set. This process was until the selected features were stabilized and no more variables were eliminated through backwards elimination.

Logistic Regression. Logistic regression (LR) models were built on the final features selected from performing Random Forest backwards elimination recursively. The glmnet package in R was used in order to perform logistic regression modeling (Friedman et al., Journal of Statistical Software. 33(1):1-22, 2010).

Internal Validation. AUCs were calculated using the pROC package in R, and 95% confidence intervals of the AUC were calculated using the DeLong method (Robin et al., BMC Bioinformatics, 12:77, 2011; DeLong et al., Biometrics. 44(3)837-845, 1988). AUCs were calculated by combining predictions across test sets using their corresponding training model. Receiver operating characteristic (ROC) curves were plotted for each model.

Results. All 73 enrolled patients were male, with a median age of 22. Nine patients (12.3%) developed VTE after enrollment, including four (5.5%) with DVT, four (5.5%) with PE, and one (1.4%) with both DVT and PE. Most of the patients suffered from blast-induced wounds (63 out of 73 patients or 86.3%). Median injury severity score (ISS) was 16, and approximately a fifth of patients had lower extremity wounds (15 out of 73 patients or 20.5%).

Random Forest modeling required three to four iterations of backwards elimination for each data set and threshold combination. Between two to ten final variables were selected for each final model. The Random Forest model with the highest area under the curve (AUC) was modeled using the clinical and serum data set and had an AUC of 0.946 [95% CI: 0.932, 0.959], sensitivity of 0.992, specificity of 0.838, and five final variables of Interleukin-15 (IL-15), Monokine Induced by Gamma (MIG), Vascular Endothelial Growth Factor (VEGF), total blood products transfused within first 24-hours, and presence of a soft tissue injury. The Random Forest serum-only model with the highest AUC had metrics of an AUC of 0.885 [95% CI: 0.870, 0.900], sensitivity of 0.794, specificity of 0.801, and four final variables of Interferon-α (IFN-α), IL-15, MIG, and VEGF. The Random Forest clinical-only model with the highest AUC had metrics of an AUC of 0.876 [95% CI: 0.854, 0.898], sensitivity of 0.980, specificity of 0.769, and four final variables of units of fresh frozen plasma (FFP) transfused within first 24-hours, total blood products transfused within first 24-hours, presence of an open fracture, and presence of a soft tissue injury.

Logistic regression models underperformed most of the Random Forest models. The logistic regression model with the highest AUC had an AUC of 0.746 [95% CI: 0.724, 0.769], sensitivity of 0.644, and specificity of 0.701, and was modeled on four clinical variables which included units of FFP transfused at initial resuscitation, total blood products transfused at initial resuscitation, presence of an open fracture, and presence of a soft tissue injury.

Sensitivities and specificities are reported at the threshold where their product is maximized, and 95% confidence intervals for the AUCs as calculated by the DeLong method are also reported.

What is claimed is:

1. A method for generating a machine learning model predicting venous thromboembolism for a subject comprising:
generating a training database storing first values of a plurality of clinical parameters and venous thromboembolism outcomes associated with a plurality of first subjects;
formatting the database into model features configured to be input into the machine learning model;
executing a feature selection algorithm to select a subset of model parameters from the plurality of clinical parameters for the machine learning model;
inputting the selected subset of features into the machine learning models for predicting venous thromboembolism;
generating, utilizing at least the machine learning models for predicting venous thromboembolism, output data indicating a prediction for venous thromboembolism; and
calculating a performance metric associated with the machine learning model in accordance with the prediction of venous thromboembolism.

2. The method of claim 1, further comprising pre-processing data that is stored in the training database including:
determining that a first value of at least one of the plurality of clinical parameters is missing;
estimating a reference value for the at least one of the plurality of clinical parameters that is missing; and
storing the reference value as the first value of the at least one of the plurality of clinical parameters in the training database.

3. The method of any one of claims 1 and 2, wherein the plurality of feature selection machine learning models comprise at least one of unsupervised machine learning algorithm, supervised machine learning algorithm, univariate t-tests, backwards elimination, and recursive feature elimination.

4. The method of any one of claims 1-3, wherein the machine learning model for predicting venous thromboembolism comprises a random forest model.

5. The method of any one of claims 1-4, further comprising:
cross-validating performances of the machine learning model, wherein cross-validating comprises iterations of leave-one-pair-out cross validation.

6. The method of any one of claims 1-5, wherein the performance metric associated with the machine learning models includes at least one of area under the curve, sensitivity, specificity, and convergence.

7. The method of any one of claims 1-6, wherein the plurality of clinical parameters comprise one or more of subject data, administration of blood products data, and injury severity data.

8. The method of any one of claims 1-7, wherein
biological data comprises one or more level of interleukin-1α (IL-1α) in a sample from the subject, level of interleukin-Iβ (IL-Iβ) in a sample from the subject, level of interleukin-1 receptor agonist (IL-1RA) in a sample from the subject, level of interleukin-2 (IL-2) in a sample from the subject, level of interleukin-2 receptor (IL-2R) in a sample from the subject, level of interleukin-3 (IL-3) in a sample from the subject, level of interleukin-4 (IL-4) in a sample from the subject, level of interleukin-5 (IL-5) in a sample from the subject, level of interleukin-6 (IL-6) in a sample from the subject, level of interleukin-7 (IL-7) in a sample from the subject, level of interleukin-8 (IL-8) in a sample from the subject, level of interleukin-10 (IL-10) in a sample from the subject, level of interleukin-12 (IL-12) in a sample from the subject, level of interleukin-13 (IL-13) in a sample from the subject, level of interleukin-15 (IL-15) in a sample from the subject, level of interleukin-17 (IL-17) in a sample from the subject, level of tumor necrosis factor alpha (TNF-α) in a sample from the subject, level of granulocyte colony stimulating factor (G-CSF) in a sample from the subject, level of granulocyte macrophage colony stimulating factor (GM-CSF) in a sample from the subject, level of interferon alpha (IFN-α) in a sample from the subject, level of interleukin-8 (IL-8) in a sample from the subject, level of interleukin-10 (IL-10) in a sample from the subject, level of interferon gamma (IFN-γ) in a sample from the subject, level of epithelial growth factor (EGF) in a sample from the subject, level of basic epithelial growth factor (bFGF) in a sample from the subject, level of hepatocyte growth factor (HGF) in a sample from the subject, level of vascular endothelial growth factor (VEGF) in a sample from a subject, the level of monocyte chemoattractant protein-1 (CCL2/MCP-1) in a sample from a subject, level of macrophage inflammatory protein-1 alpha (CCL3/MIP-Ia) in a sample from a subject, level of macrophage inflammatory protein-1 beta (CCL4/MIP-Iβ) in a sample from a subject, level of CCL5/RANTES in a sample from a subject, the level of CCL11/eotaxin in a sample from a subject, level of monokine induced by gamma interferon (CXCL9/MIG) in a sample from a subject, level of interferon gamma-induced protein-10 (CXCL10/I P10) in a sample from a subject, level of basic fibroblast growth factor (bFGF) in a sample from a subject, level of mitochondrial DNA (mtDNA) in a sample from a subject, level of soluble CD40 ligand (sCD40L) in a subject, or level of transglutaminase 2 in a sample from a subject;
subject data comprises one or more of gender, age, date of injury, length of hospital stay, length of intensive care unit (ICU) stay, number of days on a ventilator, disposition from hospital, development of nosocomial infections, sequential organ failure assessment (SOFA), injury GCS score, Marshall Classification 2 (mild diffuse injury), midline shift based on Roterdam computed tomography, temperature, arterial pH, potassium score, vascular injury score, pulse rate, or FiO₂;
administration of blood products data comprises one or more of an amount of whole blood cells administered to the subject, amount of red blood cells (RBCs) administered to the subject, amount of packed red blood cells (PRBCs) administered to the subject, amount of platelets administered to the subject, units of blood products transfused in the first 24 hours, summation of all blood transfusion products administered to the subject, or a level of total packed RBCs; and
injury severity data comprises one or more of Injury Severity Score (ISS), Abbreviated injury scale (AIS) of abdomen, AIS of chest (thorax), AIS of extremity, AIS of face, AIS of head, or AIS of skin, location of injury, presence of abdominal injury, mechanism of injury, wound depth, wound surface area, number of wound debridements, associated injuries, type of wound closure, success of wound closure, wound presence and location, compound fracture, soft tissue injury, and limb amputation.

9. The method of any one of claims 1-8, wherein the biological data comprises level of IL-15 in a sample from a subject, level of MIG in a sample from a subject, and level of VEGF in a sample from a subject.

10. The method of any one of claims 1-9, wherein the clinical parameters comprises units of total blood products transfused in the first 24 hours and soft tissue injury.

11. A method for predicting venous thromboembolism for a subject comprising:
receiving, from a second subject, a second value of at least one clinical parameter of a plurality of clinical parameters;
executing a pre-trained model for predicting venous thromboembolism, wherein the model is pre-trained by performing operations comprising:
generating a training database storing first values of a plurality of clinical parameters and venous thromboembolism associated with a plurality of first subjects;
formatting the database into model features configured to be input into the machine learning model;
executing a feature selection algorithm to select a subset of model parameters from the plurality of clinical parameters for the machine learning model;
inputting the selected subset of features into the machine learning models for predicting venous thromboembolism;
generating, utilizing at least the machine learning models for predicting venous thromboembolism, output data indicating a prediction for venous thromboembolism; and
calculating a performance metric associated with the machine learning model in accordance with the prediction of venous thromboembolism; and
outputting the predicted venous thromboembolism of the second subject.

12. The method of claim 11, further comprising pre-processing data that is stored in the training database including:
determining that a first value of at least one of the plurality of clinical parameters is missing;
estimating a reference value for the at least one of the plurality of clinical parameters that is missing; and
storing the reference value as the first value of the at least one of the plurality of clinical parameters in the training database.

13. The method of any one of claims 11 and 12, wherein the plurality of feature selection machine learning models comprise at least one of unsupervised machine learning algorithm, supervised machine learning algorithm, univariate t-tests, backwards elimination, and recursive feature elimination.

14. The method of any one of claims 11-13, wherein the machine learning model for predicting venous thromboembolism comprises a random forest model.

15. The method of any one of claims 11-14, further comprising:
cross-validating performances of the machine learning model, wherein cross-validating comprises iterations of leave-one-pair-out cross validation.

16. The method of any one of claims 11-15, wherein the performance metric associated with the machine learning models includes at least one of area under the curve, sensitivity, specificity, and convergence.

17. The method of any one of claims 11-16, wherein the plurality of clinical parameters comprise one or more of subject data, administration of blood products data, and injury severity data.

18. The method of any one of claims 11-17, wherein
biological data comprises one or more level of interleukin-1α (IL-1α) in a sample from the subject, level of interleukin-Iβ (IL-Iβ) in a sample from the subject, level of interleukin-1 receptor agonist (IL-1RA) in a sample from the subject, level of interleukin-2 (IL-2) in a sample from the subject, level of interleukin-2 receptor (IL-2R) in a sample from the subject, level of interleukin-3 (IL-3) in a sample from the subject, level of interleukin-4 (IL-4) in a sample from the subject, level of interleukin-5 (IL-5) in a sample from the subject, level of interleukin-6 (IL-6) in a sample from the subject, level of interleukin-7 (IL-7) in a sample from the subject, level of interleukin-8 (IL-8) in a sample from the subject, level of interleukin-10 (IL-10) in a sample from the subject, level of interleukin-12 (IL-12) in a sample from the subject, level of interleukin-13 (IL-13) in a sample from the subject, level of interleukin-15 (IL-15) in a sample from the subject, level of interleukin-17 (IL-17) in a sample from the subject, level of tumor necrosis factor alpha (TNF-α) in a sample from the subject, level of granulocyte colony stimulating factor (G-CSF) in a sample from the subject, level of granulocyte macrophage colony stimulating factor (GM-CSF) in a sample from the subject, level of interferon alpha (IFN-α) in a sample from the subject, level of interleukin-8 (IL-8) in a sample from the subject, level of interleukin-10 (IL-10) in a sample from the subject, level of interferon gamma (IFN-γ) in a sample from the subject, level of epithelial growth factor (EGF) in a sample from the subject, level of basic epithelial growth factor (bFGF) in a sample from the subject, level of hepatocyte growth factor (HGF) in a sample from the subject, level of vascular endothelial growth factor (VEGF) in a sample from a subject, the level of monocyte chemoattractant protein-1 (CCL2/MCP-1) in a sample from a subject, level of macrophage inflammatory protein-1 alpha (CCL3/MIP-Ia) in a sample from a subject, level of macrophage inflammatory protein-1 beta (CCL4/MIP-Iβ) in a sample from a subject, level of CCL5/RANTES in a sample from a subject, the level of CCL11/eotaxin in a sample from a subject, level of monokine induced by gamma interferon (CXCL9/MIG) in a sample from a subject, level of interferon gamma-induced protein-10 (CXCL10/I P10) in a sample from a subject, level of basic fibroblast growth factor (bFGF) in a sample from a subject, level of mitochondrial DNA (mtDNA) in a sample from a subject, level of soluble CD40 ligand (sCD40L) in a subject, or level of transglutaminase 2 in a sample from a subject;
subject data comprises one or more of gender, age, date of injury, length of hospital stay, length of intensive care unit (ICU) stay, number of days on a ventilator, disposition from hospital, development of nosocomial infections, sequential organ failure assessment (SOFA), injury GCS score, Marshall Classification 2 (mild diffuse injury), midline shift based on Roterdam computed tomography, temperature, arterial pH, potassium score, vascular injury score, pulse rate, or FiO₂;
administration of blood products data comprises one or more of an amount of whole blood cells administered to the subject, amount of red blood cells (RBCs) administered to the subject, amount of packed red blood cells (PRBCs) administered to the subject, amount of platelets administered to the subject, units of blood products transfused in the first 24 hours, summation of all blood transfusion products administered to the subject, or a level of total packed RBCs; and
injury severity data comprises one or more of Injury Severity Score (ISS), Abbreviated injury scale (AIS) of abdomen, AIS of chest (thorax), AIS of extremity, AIS of face, AIS of head, or AIS of skin, location of injury, presence of abdominal injury, mechanism of injury, wound depth, wound surface area, number of wound debridements, associated injuries, type of wound closure, success of wound closure, wound presence and location, compound fracture, soft tissue injury, and limb amputation.

19. The method of any one of claims 11-18, wherein the biological data comprises level of IL-15 in a sample from a subject, level of MIG in a sample from a subject, and level of VEGF in a sample from a subject.

20. The method of any one of claims 11-19, wherein the clinical parameters comprises units of total blood products transfused in the first 24 hours and soft tissue injury.

21. A system for predicting venous thromboembolism in a subject comprising:
one or more processors;
a memory;
a communication platform;
a training database configured to store first values of a plurality of clinical parameters and venous thromboembolism outcomes associated with a plurality of first subjects; and
a machine learning engine configured to:
format the database into model features configured to be input into a machine learning model;
execute a feature selection algorithm to select a subset of model parameters from the plurality of clinical parameters for the machine learning model;
input the selected subset of features into the machine learning models for predicting venous thromboembolism;
generate, utilizing at least the machine learning models for predicting venous thromboembolism, output data indicating a prediction for venous thromboembolism;
calculate a performance metric associated with the machine learning model in accordance with the prediction of venous thromboembolism;
output a trained machine learning model for predicting venous thromboembolism;
receive, from a second subject, a second value of at least one clinical parameter of a plurality of clinical parameters;
execute the trained model for predicting venous thromboembolism; and
output data indicating a prediction for venous thromboembolism on a display device.

22. The system of claim 21, further comprising pre-processing data that is stored in the training database including:
determining that a first value of at least one of the plurality of clinical parameters is missing;
estimating a reference value for the at least one of the plurality of clinical parameters that is missing; and
storing the reference value as the first value of the at least one of the plurality of clinical parameters in the training database.

23. The system of any one of claims 21 and 22, wherein the plurality of feature selection machine learning models comprise at least one of unsupervised machine learning algorithm, supervised machine learning algorithm, univariate t-tests, backwards elimination, and recursive feature elimination.

24. The system of any one of claims 21-23, wherein the machine learning model for predicting venous thromboembolism comprises a random forest model.

25. The system of any one of claims 21-24, further comprising:
cross-validating performances of the machine learning model, wherein cross-validating comprises iterations of leave-one-pair-out cross validation.

26. The system of any one of claims 21-25, wherein the performance metric associated with the machine learning models includes at least one of area under the curve, sensitivity, specificity, and convergence.

27. The system of any one of claims 21-26, wherein the plurality of clinical parameters comprise one or more of subject data, administration of blood products data, and injury severity data.

28. The system of any one of claims 21-27, wherein
biological data comprises one or more level of interleukin-1α (IL-1α) in a sample from the subject, level of interleukin-Iβ (IL-Iβ) in a sample from the subject, level of interleukin-1 receptor agonist (IL-1RA) in a sample from the subject, level of interleukin-2 (IL-2) in a sample from the subject, level of interleukin-2 receptor (IL-2R) in a sample from the subject, level of interleukin-3 (IL-3) in a sample from the subject, level of interleukin-4 (IL-4) in a sample from the subject, level of interleukin-5 (IL-5) in a sample from the subject, level of interleukin-6 (IL-6) in a sample from the subject, level of interleukin-7 (IL-7) in a sample from the subject, level of interleukin-8 (IL-8) in a sample from the subject, level of interleukin-10 (IL-10) in a sample from the subject, level of interleukin-12 (IL-12) in a sample from the subject, level of interleukin-13 (IL-13) in a sample from the subject, level of interleukin-15 (IL-15) in a sample from the subject, level of interleukin-17 (IL-17) in a sample from the subject, level of tumor necrosis factor alpha (TNF-α) in a sample from the subject, level of granulocyte colony stimulating factor (G-CSF) in a sample from the subject, level of granulocyte macrophage colony stimulating factor (GM-CSF) in a sample from the subject, level of interferon alpha (IFN-α) in a sample from the subject, level of interleukin-8 (IL-8) in a sample from the subject, level of interleukin-10 (IL-10) in a sample from the subject, level of interferon gamma (IFN-γ) in a sample from the subject, level of epithelial growth factor (EGF) in a sample from the subject, level of basic epithelial growth factor (bFGF) in a sample from the subject, level of hepatocyte growth factor (HGF) in a sample from the subject, level of vascular endothelial growth factor (VEGF) in a sample from a subject, the level of monocyte chemoattractant protein-1 (CCL2/MCP-1) in a sample from a subject, level of macrophage inflammatory protein-1 alpha (CCL3/MIP-Ia) in a sample from a subject, level of macrophage inflammatory protein-1 beta (CCL4/MIP-Iβ) in a sample from a subject, level of CCL5/RANTES in a sample from a subject, the level of CCL11/eotaxin in a sample from a subject, level of monokine induced by gamma interferon (CXCL9/MIG) in a sample from a subject, level of interferon gamma-induced protein-10 (CXCL10/I P10) in a sample from a subject, level of basic fibroblast growth factor (bFGF) in a sample from a subject, level of mitochondrial DNA (mtDNA) in a sample from a subject, level of soluble CD40 ligand (sCD40L) in a subject, or level of transglutaminase 2 in a sample from a subject;
subject data comprises one or more of gender, age, date of injury, length of hospital stay, length of intensive care unit (ICU) stay, number of days on a ventilator, disposition from hospital, development of nosocomial infections, sequential organ failure assessment (SOFA), injury GCS score, Marshall Classification 2 (mild diffuse injury), midline shift based on Roterdam computed tomography, temperature, arterial pH, potassium score, vascular injury score, pulse rate, or FiO₂;
administration of blood products data comprises one or more of an amount of whole blood cells administered to the subject, amount of red blood cells (RBCs) administered to the subject, amount of packed red blood cells (PRBCs) administered to the subject, amount of platelets administered to the subject, units of blood products transfused in the first 24 hours, summation of all blood transfusion products administered to the subject, or a level of total packed RBCs; and
injury severity data comprises one or more of Injury Severity Score (ISS), Abbreviated injury scale (AIS) of abdomen, AIS of chest (thorax), AIS of extremity, AIS of face, AIS of head, or AIS of skin, location of injury, presence of abdominal injury, mechanism of injury, wound depth, wound surface area, number of wound debridements, associated injuries, type of wound closure, success of wound closure, wound presence and location, compound fracture, soft tissue injury, and limb amputation.

29. The system of any one of claims 21-28, wherein the biological data comprises level of IL-15 in a sample from a subject, level of MIG in a sample from a subject, and level of VEGF in a sample from a subject.

30. The system of any one of claims 21-29, wherein the clinical parameters comprises units of total blood products transfused in the first 24 hours and soft tissue injury.

31. A method of predicting venous thromboembolism in a subject comprising:
obtaining a biological sample from the subject;
measuring IL-15, MIG, and VEGF from the biological sample; and
predicting venous thromboembolism in the subject, based at least in part on levels of IL-15, MIG, and VEGF.

32. The method of claim 31, wherein the method further comprises measuring total number of blood products transfused in first 24 hours.

33. The method of claim 31 or claim 32, wherein the method further comprises assessing soft tissue injury.

34. The method of any one of claims 31-33, wherein the method further comprises treating the subject for venous thromboembolism.
